(19) United States
(12) Patent Application Publication
(10) Pub. No.: US 2020/0352921 A1
(43) Pub. Date: Nov. 12, 2020

(54) ARYL-SULFONAMIDE AND ARYL-SULFONE DERIVATIVES AS TRPML MODULATORS

(71) Applicant: CalyGene Biotechnolgy, Inc., Camden, DE (US)

(72) Inventor: Congxin Liang, Palm Beach Gardens, FL (US)

(21) Appl. No.: 16/611,323

(22) PCT Filed: May 6, 2018
(86) PCT No.: PCT/US2018/031283

§ 371 Date: Nov. 6, 2019

Related U.S. Application Data
(60) Provisional application No. 62/502,750, filed on May 7, 2017.

(51) Int. Cl. A61K 31/445; A61K 31/5375; A61K 31/4535; C07D 409/12; C07D 295/26; C07D 211/96; C07D 401/06; C07D 401/12; A61K 31/4545; A61K 31/4709; A61K 31/47; A61K 31/136; C07C 317/14; A61P 1/04

(52) U.S. Cl. CPC A61K 31/445; A61K 31/5375; A61K 31/4535; C07D 409/12; C07D 295/26; C07D 211/96; A61P 1/04; C07D 401/12; A61K 31/4545; A61K 31/4709; A61K 31/47; A61K 31/136; C07C 317/14; C07D 401/06

(57) ABSTRACT

The new arylsulfonamide and arylsulfone derivatives are modulators of TRPML and are useful in treating disorders related to TRPML activities and lysosome functions such as acid-related disorders and cancer.

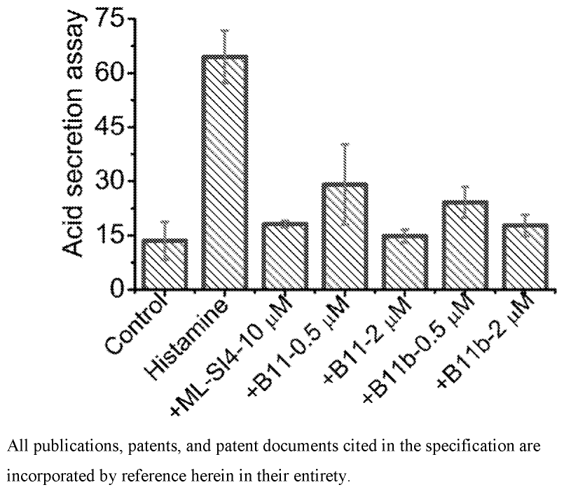

### CROSS-REFERENCE TO RELATED APPLICATIONS

This application claims the priority of U.S. provisional application Ser. No. 62/502,750, filed May 7, 2017, which is incorporated herein by reference in its entirety.

### TECHNICAL FIELD OF THE INVENTION

This invention relates to novel aryl-sulfonamide and sulfone derivatives, their salts, solvates, hydrates and polymorphs thereof as TRPML modulators. The invention also provides compositions comprising a compound of this invention and the use of such compositions in methods of treating diseases and conditions associated with TRPML and are useful in treating disorders related to TRPML activities and lysosome functions such as acid-related diseases and cancer.

### BACKGROUND OF THE INVENTION

Lysosomes are the cell's degradation center. To adapt to different environmental conditions, the cell has evolved a set of delicate mechanisms to rapidly change lysosome function, which is referred to as lysosomal adaptation. Notably, lysosomal adaptation is required for cell survival under low nutrient conditions, and thus might be a target for cancer treatment (Piao S, Amaravadi R K. 2016. Targeting the lysosome in cancer. PMID: 26599426). TRPML1, a lysosomal Ca2+-permeant ion channel, is an essential player required for lysosomal adaptation. The activity of TRPML1 is potently (up to 10-fold) and rapidly increased upon nutrient starvation. Furthermore, pharmacological inhibition or genetic deletion of TRPML1 completely abolished the effects of starvation on boosting the degradation capability of lysosomes.

Because lysosome storage is also seen in common neurodegenerative diseases such as Alzheimer's and Parkinson's, understanding the mechanisms underlying the positive feedback loop may provide therapeutic approaches not only for lysosome storage diseases (LSDs), but also for common sporadic neurodegenerative diseases. A lysosome-localized Ca²⁺ channel, TRPML1, has been recently identified as a key regulator of most membrane trafficking processes in the lysosome. Human mutations of TRPML1 can lead to lysosomal trafficking defects, lysosome storage, and neurodegenerative diseases.

TRPML1 (abbreviated as ML1), a member of the TRP-type Ca²⁺ channel superfamily, is the principle Ca²⁺ channel in the lysosome [see e.g. Cheng, X., et al., Mucolipins: Intracellular TRPML1-3 channels. FEBS Lett, 2010. 584(10): p. 2013-21]. Loss-of-function mutations in the human TRPML1 gene cause Type IV Mucolipidosis (ML4), a lysosome storage neurodegenerative disease. TRPML1^{-/-} (abbreviated as ML1^{-/-}) skin fibroblasts from ML4 patients are characterized by the accumulation of enlarged endosomal/lysosomal compartments (vacuoles) in which lipids and other biomaterials build up, suggestive of trafficking defects. Analyses of trafficking kinetics suggest that the primary defects are in the late endocytic pathways. First, ML1 is likely to be required for the formation of transport vesicles from the LEL to the Trans-Golgi Network (TGN) (LEL-to-TGN retrograde trafficking). Second, fusion of lysosomes with the plasma membrane (referred to as lysosomal exocytosis), a process that is important in cellular waste elimination and membrane repair, is defective in ML4 cells. Defects in either trafficking steps could lead to lysosome storage. Because the release of Ca²⁺ from lysosomes (lysosomal Ca²⁺ release) is essential for both trafficking steps, it is hypothesize that ML1 is indeed the Ca²⁺ release channel that regulates lysosomal trafficking.

PI(3,5)P2 is a low-abundance phosphoinositide, is the primary activator of ML1, and a positive regulator of lysosomal trafficking. Both TRPML1-lacking and PI(3,5)P2-deficient cells exhibit defects in LEL-to-Golgi retrograde trafficking and autophagosome-lysosome fusion, suggesting that the TRPML1-PI(3,5)P2 system represents a common signaling pathway essential for late endocytic trafficking.

TRPML1 also plays an essential role in autophagy. TRPML1 activation leads to lysosomal Ca2+ release, TFEB-nuclear translocation, and increases of LC3-II expression and autophagy (Zhang X, Cheng X, Yu L, Yang J, Calvo R, Patnaik S, Hu X, Gao Q, Yang M, Lawas M, Delling M, Marugan J, Ferrer M, and Xu H. 2016, PMID: 27357649). In addition, inhibition of TRPML1 impairs ROS-induced autophagy and blocks the clearance of damaged mitochondria and removal of excess ROS (Zhang X, Cheng X, Yu L, Yang J, Calvo R, Patnaik S, Hu X, Gao Q, Yang M, Lawas M, Delling M, Marugan J, Ferrer M, and Xu H. 2016, PMID: 27357649).

### SUMMARY OF THE INVENTION

The invention relates to aryl-sulfonamide and sulfone compounds, compositions comprising the compounds, and methods of using the compounds and compound compositions. The compounds and compositions comprising them are useful for treating or preventing disease or disease symptoms, including those mediated by or associated with TRPMLs. Modulating TRPML1 activity may provide novel therapeutic approaches to treat acid-related diseases and cancer.

In various embodiments, the invention provides a TRPML modulatory compound of formula (I):

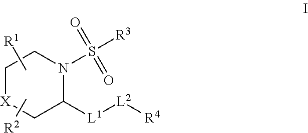

wherein:

R¹ and R² each are independently H, alkyl, haloalkyl, halogen, oxo, amino, or alkylamino; or R¹ and R² together with the atoms they are bonded form a 5-7 membered aryl, heteroaryl, cycloalkyl, cycloheteroalkyl or partially unsaturated ring optionally substituted with one or more substituents independently selected from the group consisting of halo, cyano, (C₁-C₆)alkyl, (C₁-C₆)haloalkyl, (C₂-C₆)alkenyl, (C₂-C₆)alkynyl, (C₁-C₆)alkoxy, and (C₁-C₆)haloalkoxy;

R³ and R⁴ are each independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, or two substituents together with the atoms they are bonded form a 5-7 membered cycloalkyl, or cycloheteroalkyl optionally substituted with one or more substituents independently selected from the group consisting of halo, and (C₁-C₆)alkyl; wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;

X is CR⁶R⁷, O, SOq wherein q is 0, 1 or 2, or NR⁶; R⁶ and R⁷ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR; R is an (C₁-C₆)alkyl.

In various embodiments, the invention provides a TRPML modulatory compound of formula (II):

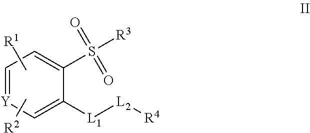

or a salt thereof, or a prodrug, or a salt of a prodrug thereof, or a hydrate, solvate, or polymorph thereof, wherein:

R¹ and R² each are independently H, alkyl, haloalkyl, alkoxy, heteroalkoxy, halogen, oxo, amino, or alkylamino; or R¹ and R² together with the atoms they are bonded form a 5-7 membered aryl, heteroaryl, cycloalkyl or partially unsaturated ring optionally substituted with one or more substituents independently selected from the group consisting of halo, cyano, (C₁-C₆)alkyl, (C₁-C₆)haloalkyl, (C₂-C₆)alkenyl, (C₂-C₆)alkynyl, (C₁-C₆)alkoxy, and (C₁-C₆)haloalkoxy;

R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl

Y is N or CR⁶; R⁶ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR; R is an (C₁-C₆)alkyl.

In various embodiments, the invention provides a method for modulating TRPLMs in a mammal, comprising administering to the mammal an effective amount of a compound of the invention.

In various embodiments, the invention provides a method for treating a condition in a mammal, wherein modulation of TRPMLs is medically indicated, comprising administering to the mammal an effective amount of a compound of the invention. The condition can be an acid-related disorder, more specifically the condition can be a gastric disorder.

In various embodiments, the invention provides a method for treating an acid-related disorder in a mammal, comprising administering to the mammal an effective amount of a compound of the invention and another agent. The other agent can be a proton pump inhibitor.

In various embodiments, the invention provides a method for treating a condition in a mammal, wherein abnormal lysosome function is medically indicated, comprising administering to the mammal an effective amount of a compound of the invention. The condition can be cancer.

In various embodiments, the invention provides a method for treating an acid-related disorder using a TRPML inhibitor.

In various embodiments, the invention provides a method for modulating tubulovesicle and lysosome functions in a mammal, comprising administering to the mammal an effective amount of a TRPML inhibitor.

In various embodiments, the invention provides a method for treating a condition in a mammal, wherein abnormal functioning of lysosomes is medically indicated, comprising administering to the mammal an effective amount of a TRPML inhibitor.

### DETAILED DESCRIPTION OF THE INVENTION

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

As used herein, “individual” (as in the subject of the treatment) or “patient” means both mammals and non-mammals. Mammals include, for example, humans; non-humanprimates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein TRPMLs play a role in the biochemical mechanisms involved in the disease or medical condition or symptom(s) thereof such that a therapeutically beneficial effect can be achieved by acting on TRPMLs, e.g. with an effective amount or concentration of a synthetic ligand of the invention. “Acting on” TRPMLs, or “modulating” TRPMLs, can include binding to TRPMLs and/or inhibiting the bioactivity of TRPMLs and/or allosterically regulating the bioactivity of TRPMLs in vivo.

The expression “effective amount”, when used to describe therapy to an individual suffering from a disorder, refers to the quantity or concentration of a compound of the invention that is effective to inhibit or otherwise act on TRPMLs in the individual's tissues wherein TRPMLs involved in the disorder, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents, or provides prophylaxis for, the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

It is to be further understood that where descriptions of various embodiments use the term “comprising,” those skilled in the art would understand that in some specific instances, an embodiment can be alternatively described using language “consisting essentially of” or “consisting of.”

By “chemically feasible” is meant a bonding arrangement or a compound where the generally understood rules of organic structure are not violated; for example a structure within a definition of a claim that would contain in certain situations, e.g., a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All single enantiomer, diastereomeric, and racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, bounded by the understanding of the person of ordinary skill as to the size of the group as being reasonable for a molecular entity; and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded, as the number of atoms in the group cannot be infinite and is limited by ordinary understanding.

It has become widely recognized by those skilled in the art that the incorporation of isotopic forms of an atom may impart useful properties. For example, a deuterium atom (²H) may be specifically introduced in place of a hydrogen atom which would otherwise represent the natural distribution of hydrogen isotopes, mostly ¹H. The use of one or more such isotopic substitutions may alter the properties of the resultant composition, including alterations in relevant properties in a treated animal, such as a longer half-life or duration of action of the composition. The isotope may also enable methods to detect the amount of the composition in affected tissue, such as by detection of radiation from isotopes such as ³H and ¹⁴C. Chemical methods for incorporating isotopes (examples including, but not limited to, ²H, ³H, ¹³C, ¹⁴C) are well-known in the art and the claims of this invention encompass such isotopic forms.

In general, “substituted” refers to an organic group as defined herein in which one or more bonds to a hydrogen atom contained therein are replaced by one or more bonds to a non-hydrogen atom such as, but not limited to, a halogen (e.g., F, Cl, Br, or I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, nitroso groups, N-oxides, hydrazides, azides, and enamines; and other heteroatoms in various other groups. Non-limiting examples of substituents that can be bonded to a substituted carbon (or other) atom include F, Cl, Br, I, OR, CN, NO, NO₂, ONO₂, azido, CF₃, OCF₃, R, O (oxo), S (thiono), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen or a carbon-based moiety, and wherein the carbon-based moiety can itself be further substituted; for example, R can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl can be further independently mono- or multi-substituted with some or all of the above-listed groups, or with other groups; or wherein two R groups bonded to a nitrogen atom or to adjacent nitrogen atoms can together with the nitrogen atom or atoms form a heterocyclyl, which can be further mono- or independently multi-substituted with some or all of the above-listed groups, or with other groups.

In various embodiments, a substituent can be any of halo, (C1-C6)alkyl, (C1-C6)alkoxy, (C1-C6)haloalkyl, hydroxy(C1-C6)alkyl, alkoxy(C1-C6)alkyl, (C1-C6)alkanoyl, (C1-C6)alkanoyloxy, cyano, nitro, azido, R₂N, R₂NC(O), R₂NC(O)O, R₂NC(O)NR, (C1-C6)alkenyl, (C1-C6)alkynyl, (C6-C10)aryl, (C6-C10)aryloxy, (C6-C10)aroyl, (C6-C10)aryl(C1-C6)alkyl, (C6-C10)aryl(C1-C6)alkoxy, (C6-C10)aryloxy(C1-C6)alkyl, (C6-C10)aryloxy(C1-C6)alkoxy, (3- to 9-membered)heterocyclyl, (3- to 9-membered)heterocyclyl(C1-C6)alkyl, (3- to 9-membered)heterocyclyl(C1-C6)alkoxy, (5- to 10-membered)heteroaryl, (5- to 10-membered)heteroaryl(C1-C6)alkyl, (5- to 10-membered)heteroaryl(C1-C6)alkoxy, or (5- to 10-membered)heteroaroyl. For example, R independently at each occurrence can be H, (C1-C6)alkyl, or (C6-C10)aryl, wherein any alkyl or aryl group may be substituted with 0-3 substituents independently selected from, but not limited to, the above-listed groups.

In various embodiments, a substituent can be any of halo, (C1-C6)alkyl, (C1-C6)alkoxy, (C1-C6)haloalkyl, hydroxy(C1-C6)alkyl, alkoxy(C1-C6)alkyl, (C1-C6)alkanoyl, (C1-C6)alkanoyloxy, cyano, nitro, azido, R₂N, R₂NC(O), R₂NC(O)O, R₂NC(O)NR, (C1-C6)alkenyl, (C1-C6)alkynyl, (C6-C10)aryl, (C6-C10)aryloxy, (C6-C10)aroyl, (C6-C10)aryl(C1-C6)alkyl, (C6-C10)aryl(C1-C6)alkoxy, (C6-C10)aryloxy(C1-C6)alkyl, (C6-C10)aryloxy(C1-C6)alkoxy, (3- to 9-membered)heterocyclyl, (3- to 9-membered)heterocyclyl(C1-C6)alkyl, (3- to 9-membered)heterocyclyl(C1-C6)alkoxy, (5- to 10-membered)heteroaryl, (5- to 10-membered)heteroaryl(C1-C6)alkyl, (5- to 10-membered)heteroaryl(C1-C6)alkoxy, or (5- to 10-membered)heteroaroyl. For example, R independently at each occurrence can be H, (C1-C6)alkyl, or (C6-C10)aryl, wherein any alkyl or aryl group may be substituted with 0-3 substituents independently selected from, but not limited to, the above-listed groups.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxa” or “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR₂)ₙ wherein n is 1, 2, 3, or more, and each R is independently selected.

Another divalent substituent is an alkylidene carbon, represented as C═ and signifying that the carbon atom so indicated, which also bears two additional groups, is double bonded to a third group. For example, (CH₃)₂C═ indicates an isopropylidene group bonded to another carbon or nitrogen atom.

C(O) and S(O)₂ groups can also be bound to one or two heteroatoms, such as nitrogen or oxygen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a “urea.” When a C(O) is bonded to one oxygen and one nitrogen atom, the resulting group is termed a “carbamate” or “urethane.” When a S(O)₂ group is bound to one carbon and one nitrogen atom, the resulting unit is termed a “sulfonamide.” When a S(O)₂ group is bound to two nitrogen atoms, the resulting unit is termed a “sulfamide.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom, or to a substituent group as defined above. Therefore, substituted cycloalkyl, aryl, heterocyclyl and heteroaryl groups can also be substituted with alkyl, alkenyl, and alkynyl groups, or with the substituent groups listed above or other substituent groups know to persons of ordinary skill in the art.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. Ring systems can be mono- or independently multi-substituted with substituents as are described above. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

When a number of carbon atoms in a group, e.g., an alkyl, alkenyl, alkynyl, cycloalkyl, aryl, etc., is specified as a range, each individual integral number representing the number of carbon atoms is intended. For example, recitation of a (C₁-C₄)alkyl group indicates that the alkyl group can be any of methyl, ethyl, propyl, isopropyl, butyl, sec-butyl, isobutyl, or tert-butyl. It is understood that a specification of a number of carbon atoms must be an integer.

When a number of atoms in a ring is specified, e.g., a 3- to 9-membered cycloalkyl or heterocyclyl ring, the cycloalkyl or heterocyclyl ring can include any of 3, 4, 5, 6, 7, 8, or 9 atoms. A cycloalkyl ring is carbocyclic; a heterocyclyl ring can include atoms of any element in addition to carbon capable of forming two or more bonds, e.g., nitrogen, oxygen, sulfur, and the like. The number of atoms in a ring is understood to necessarily be an integer.

Alkyl groups include straight chain and branched carbon-based groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the substituent groups listed above, for example, amino, hydroxy, cyano, carboxy, nitro, thio, alkoxy, and halogen groups. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

Cycloalkyl groups are groups containing one or more carbocyclic ring including, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above.

Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group, i.e., a cycloalkyl including one or more carbon-carbon double bond.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

Aryl groups are cyclic aromatic hydrocarbons that do not contain heteroatoms in the ring. An aromatic compound, as is well-known in the art, is a multiply-unsaturated cyclic system that contains 4n+2π electrons where n is an integer. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl, also termed arylalkyl, groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups are defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which one or more ring atom is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. Ring sizes can also be expressed by the total number of atoms in the ring, e.g., a 3- to 10-membered heterocyclyl group, counting both carbon and non-carbon ring atoms. A heterocyclyl ring can also include one or more double bonds. A heteroaryl ring is an embodiment of a heterocyclyl group. The term “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The term also includes polycyclic, e.g., bicyclo- and tricyclo-ring systems containing one or more heteroatom such as, but not limited to, quinuclidyl.

Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure, which is a multiply-unsaturated cyclic system that contains 4n+2π electrons wherein n is an integer A heteroaryl group designated as a C₂-heteroaryl can be a 5-ring (i.e., a 5-membered ring) with two carbon atoms and three heteroatoms, a 6-ring (i.e., a 6-membered ring) with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms.

The term “alkoxy” or “alkoxyl” refers to an oxygen atom connected to an alkyl group, including a cycloalkyl group, as are defined above. Examples of linear alkoxy groups include but are not limited to methoxy, ethoxy, n-propoxy, n-butoxy, n-pentyloxy, n-hexyloxy, and the like. Examples of branched alkoxy include but are not limited to isopropoxy, sec-butoxy, tert-butoxy, isopentyloxy, isohexyloxy, and the like. Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The term “cycloalkoxy” as used herein refers to a cycloalkyl group attached to oxygen (cycloalkyl-O—). Examples of cyclic alkoxy include but are not limited to cyclopropyloxy, cyclobutyloxy, cyclopentyloxy, cyclohexyloxy, and the like. Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, and the like.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein allhydrogen atoms are replaced by the same or differing halogen atoms, such as fluorine and/or chlorine atoms. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention. “Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. All commercially available chemicals were obtained from Aldrich, Alfa Aesare, Wako, Acros, Fisher, Fluka, Maybridge or the like and were used without further purification, except where noted. Dry solvents are obtained, for example, by passing these through activated alumina columns.

The present invention further embraces isolated compounds of the invention. The expression “isolated compound” refers to a preparation of a compound of the invention, or a mixture of compounds the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein.

It will be understood that when compounds of the present invention contain one or more chiral centers, the compounds may exist in, and may be isolated as single and substantially pure enantiomeric or diastereomeric forms or as racemic mixtures. The present invention therefore includes any possible enantiomers, diastereomers, racemates or mixtures thereof of the compounds of the invention.

### COMPOUNDS OF THE INVENTION

In various embodiments, the invention provides a TRPML modulatory compound of formula (I):

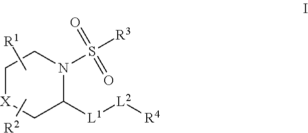

or a salt thereof, or a prodrug, or a salt of a prodrug thereof, or a hydrate, solvate, or polymorph thereof, wherein:

R¹ and R² each are independently H, alkyl, haloalkyl, halogen, oxo, amino, or alkylamino; or R¹ and R² together with the atoms they are bonded form a 5-7 membered aryl, heteroaryl, cycloalkyl, cycloheteroalkyl or partially unsaturated ring optionally substituted with one or more substituents independently selected from the group consisting of halo, cyano, (C₁-C₆)alkyl, (C₁-C₆)haloalkyl, (C₂-C₆)alkenyl, (C₂-C₆)alkynyl, (C₁-C₆)alkoxy, and (C₁-C₆)haloalkoxy;

R³ and R⁴ are each independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, or two substituents together with the atoms they are bonded form a 5-7 membered cycloalkyl, or cycloheteroalkyl optionally substituted with one or more substituents independently selected from the group consisting of halo, and (C₁-C₆)alkyl; wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;

X is CR⁶R⁷, O, SOq wherein q is 0, 1 or 2, or NR⁶; R⁶ and R⁷ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

L¹ and L² are each independently a bond, (C₁-C₃)alkyl, —O—, —NH—, —S—, —S(O)—, —S(O)₂—, —NR—, or —C(O)—, provided L and L² are not both —O—, —NH—, —S—, —S(O)—, —S(O)₂—, or —NR; R is an (C₁-C₆)alkyl.

In various embodiments, the invention provides a TRPML modulatory compound of formula (IA):

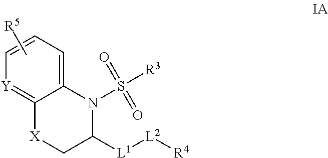

wherein R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, or two substituents together with the atoms they are bonded form a 5-7 membered cycloalkyl, or cycloheteroalkyl optionally substituted with one or more substituents independently selected from the group consisting of halo, and (C₁-C₆)alkyl; wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;

R⁵ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

X is CR⁶R⁷, O, SOq wherein q is 0, 1 or 2, or NR⁶; R⁶ and R⁷ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

Y is N or CR⁸; R′ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR; R is an (C₁-C₆)alkyl.

In various embodiments, the compound is any of those shown in Table 1 and 2.

In various embodiments, the invention provides a TRPML modulatory compound of formula (II):

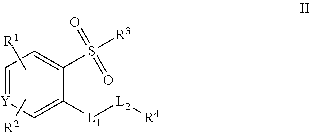

or a salt thereof, or a prodrug, or a salt of a prodrug thereof, or a hydrate, solvate, or polymorph thereof, wherein:

R¹ and R² each are independently H, alkyl, haloalkyl, alkoxy, heteroalkoxy, halogen, oxo, amino, or alkylamino; or R¹ and R² together with the atoms they are bonded form a 5-7 membered aryl, heteroaryl, cycloalkyl or partially unsaturated ring optionally substituted with one or more substituents independently selected from the group consisting of halo, cyano, (C₁-C₆)alkyl, (C₁-C₆)haloalkyl, (C₂-C₆)alkenyl, (C₂-C₆)alkynyl, (C₁-C₆)alkoxy, and (C₁-C₆)haloalkoxy;

R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═), R′₂NC(═O), R″S, R″S(O), and R″S(O)₂, wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;

Y is N or CR⁶; R⁶ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;

L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR; R is an (C₁-C₆)alkyl.

In various embodiments, the invention provides a TRPML modulatory compound of formula (IIA):

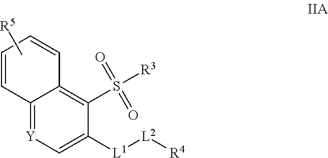

wherein R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;

Y is N or CR⁶;

L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR; R is an (C₁-C₆)alkyl;

R⁵ and R⁶ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy.

In various embodiments, the compound is any of those shown in Table 3 and 4.

Another aspect of the invention is a composition, comprising a compound of the invention, alone or in combination with another medicament. As set forth herein, compounds of the invention include stereoisomers, tautomers, pharmaceutically acceptable salt thereof, or a prodrug, or a salt of a prodrug thereof; or a hydrate, solvate, or polymorph thereof.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

### EXAMPLES

Exemplar compounds of formula (I) are shown in table 1:

TABLE 1

| | |
|---|---|
| B1 | 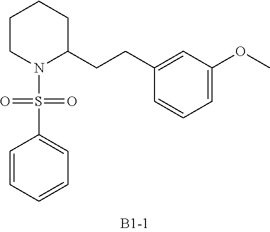 [SMILES: COc1cccc(CCC2CCCCN2S(=O)(=O)c2ccccc2)c1] |
| | 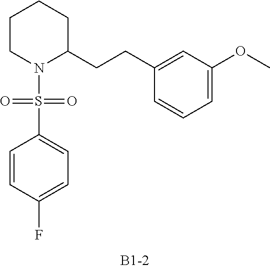 [SMILES: COc1cccc(CCC2CCCCN2S(=O)(=O)c2ccc(F)cc2)c1] |
| | 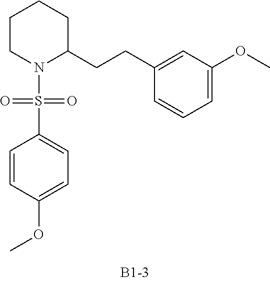 [SMILES: COc1ccc(S(=O)(=O)N2CCCCC2CCc2cccc(OC)c2)cc1] |
| | 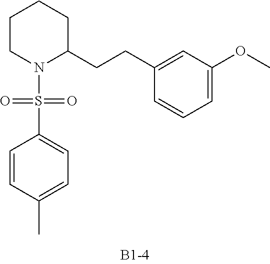 [SMILES: COc1cccc(CCC2CCCCN2S(=O)(=O)c2ccc(C)cc2)c1] |
| B2 | 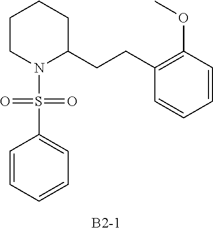 |
| | 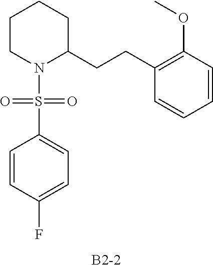 [SMILES: COc1ccccc1CCC1CCCCN1S(=O)(=O)c1ccc(F)cc1 ; "B2-2"] |
| | 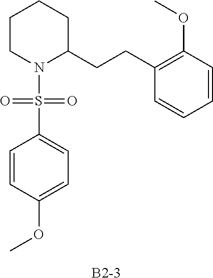 [SMILES: COc1ccc(S(=O)(=O)N2CCCCC2CCc2ccccc2OC)cc1 ; "B2-3"] |
| | 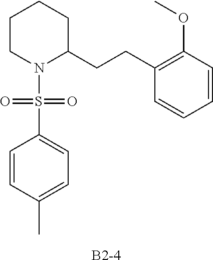 [SMILES: COc1ccccc1CCC1CCCCN1S(=O)(=O)c1ccc(C)cc1 ; "B2-4"] |
| B3 | 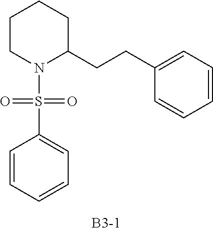 |
| | 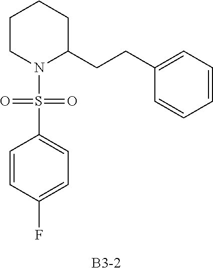 |
| | 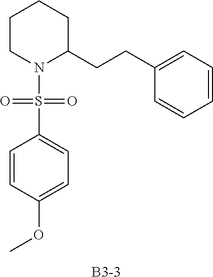 |
| | 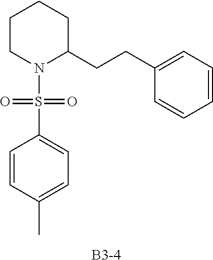 [SMILES: Cc1ccc(S(=O)(=O)N2CCCCC2CCc2ccccc2)cc1 ; "B3-4"] |
| B4 | 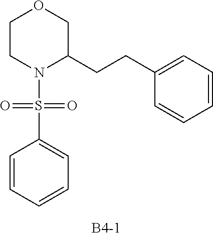 |
| | 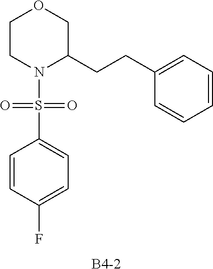 |
| | 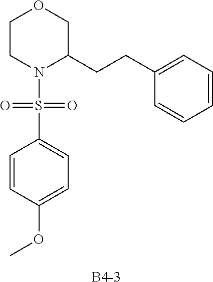 |
| | 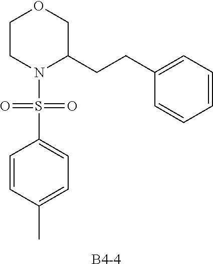 |
| B6 | 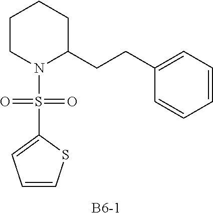 |
| | 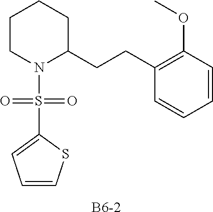 |
| B9 | 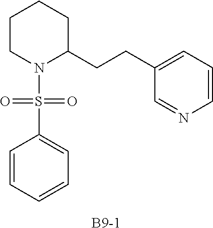 |
| | 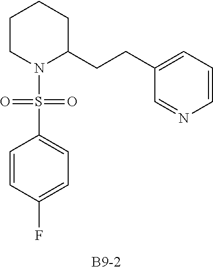 |
| | 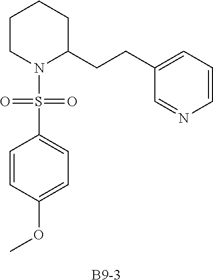 |
| | 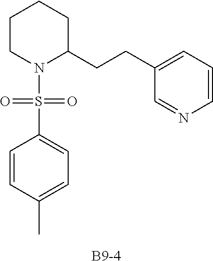 |
| B10 | 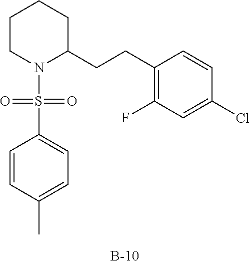 |
| | 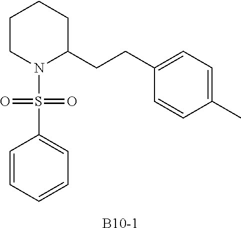 [SMILES: Cc1ccc(CCC2CCCCN2S(=O)(=O)c2ccccc2)cc1 ; "B10-1"] |
| | 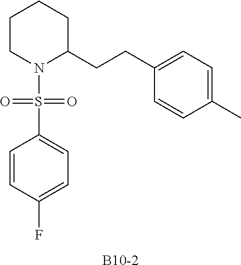 [SMILES: Cc1ccc(CCC2CCCCN2S(=O)(=O)c2ccc(F)cc2)cc1 ; "B10-2"] |
| | 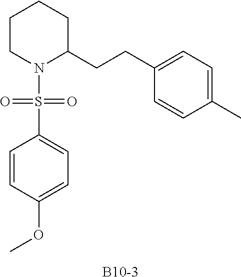 [SMILES: COc1ccc(S(=O)(=O)N2CCCCC2CCc2ccc(C)cc2)cc1 ; "B10-3"] |
| | 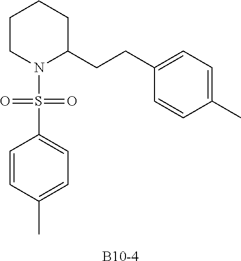 [SMILES: Cc1ccc(CCC2CCCCN2S(=O)(=O)c2ccc(C)cc2)cc1 ; "B10-4"] |
| B19 | 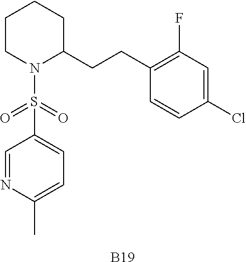 |
| B20 | 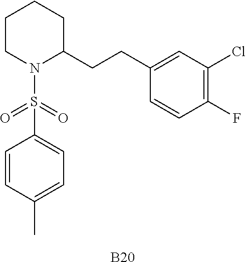 |
| B21 | 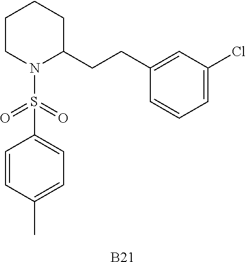 |
| B22 | 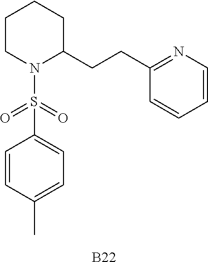 |
| B23 | 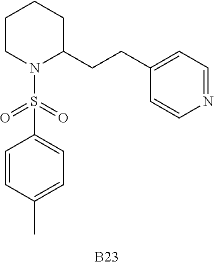 |

Exemplary compounds of formula (IA) are shown in table 2:

TABLE 2

| | |
|---|---|
| B7-4 | 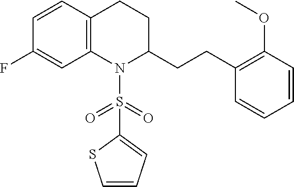 [SMILES: COc1ccccc1CCC1CCc2ccc(F)cc2N1S(=O)(=O)c1cccs1] |
| B11 | 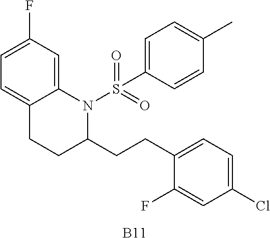 |
| | 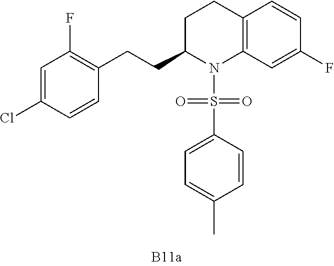 [SMILES: Cc1ccc(S(=O)(=O)N2c3cc(F)ccc3CC[C@@H]2CCc2ccc(Cl)cc2F)cc1] |
| | 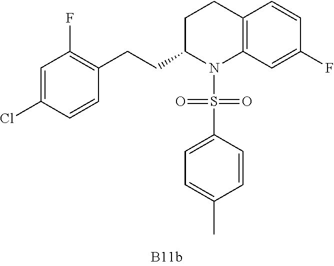 [SMILES: Cc1ccc(S(=O)(=O)N2c3cc(F)ccc3CC[C@H]2CCc2ccc(Cl)cc2F)cc1] |
| B12 | 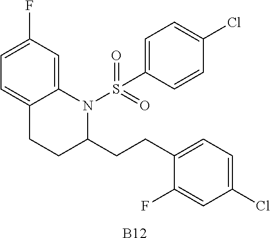 |
| B28 | 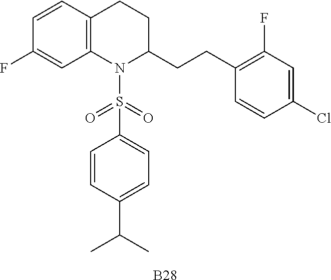 |
| B29 | 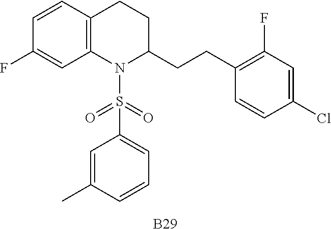 [SMILES: Cc1cccc(S(=O)(=O)N2c3cc(F)ccc3CCC2CCc2ccc(Cl)cc2F)c1 ; "B29"] |
| B30 | 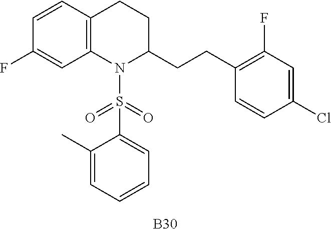 |
| B31 | 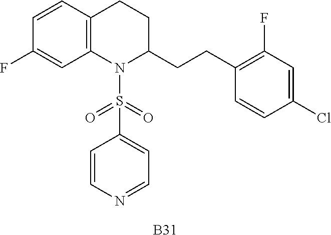 [SMILES: O=S(=O)(c1ccncc1)N1c2cc(F)ccc2CCC1CCc1ccc(Cl)cc1F ; "B31"] |
| B32 | 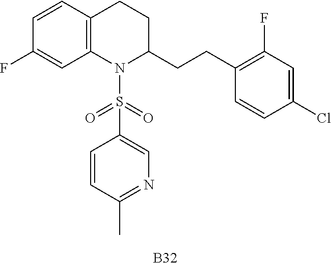 [SMILES: Cc1ccc(S(=O)(=O)N2c3cc(F)ccc3CCC2CCc2ccc(Cl)cc2F)cn1 ; "B32"] |
| B33 | 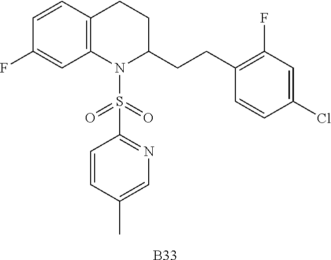 [SMILES: Cc1ccc(S(=O)(=O)N2c3cc(F)ccc3CCC2CCc2ccc(Cl)cc2F)nc1 ; "B33"] |
| B34 | 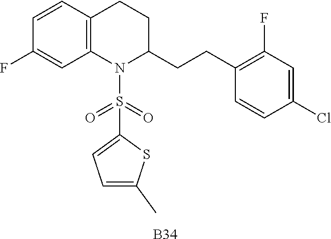 [SMILES: Cc1ccc(S(=O)(=O)N2c3cc(F)ccc3CCC2CCc2ccc(Cl)cc2F)s1 ; "B34"] |
| B40 | 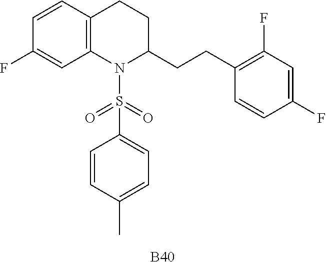 [SMILES: Cc1ccc(S(=O)(=O)N2c3cc(F)ccc3CCC2CCc2ccc(F)cc2F)cc1 ; "B40"] |
| B41 | 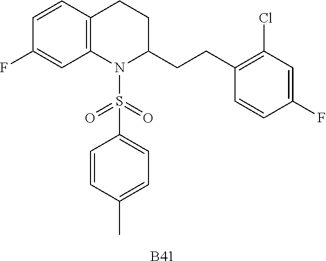 [SMILES: Cc1ccc(S(=O)(=O)N2c3cc(F)ccc3CCC2CCc2ccc(F)cc2Cl)cc1 ; "B41"] |
| B44 | 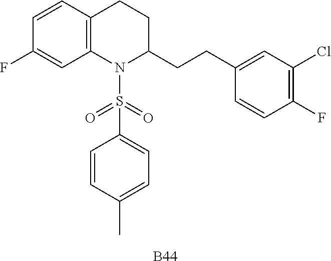 [SMILES: "B44" ; Cc1ccc(S(=O)(=O)N2c3cc(F)ccc3CCC2CCc2ccc(F)c(Cl)c2)cc1] |
| B45 | 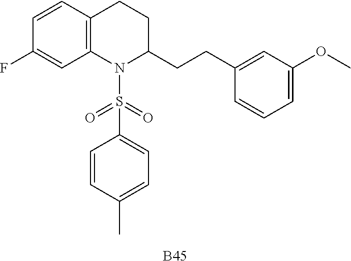 [SMILES: COc1cccc(CCC2CCc3ccc(F)cc3N2S(=O)(=O)c2ccc(C)cc2)c1 ; "B45"] |
| B46 | 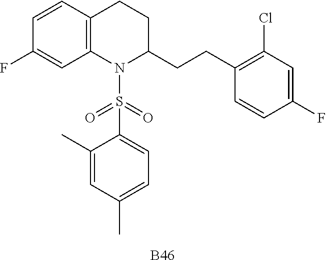 |
| B47 | 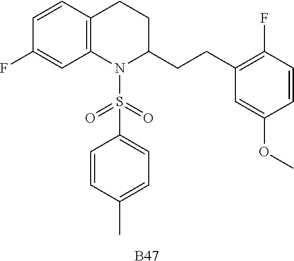 |
| B48 | 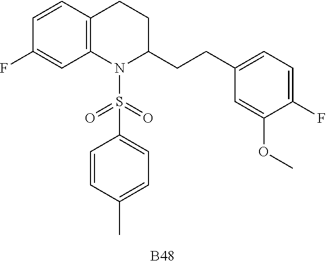 |
| B49 | 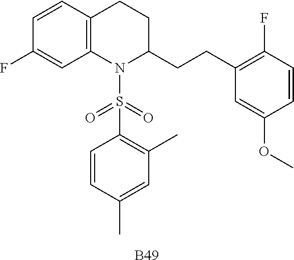 |
| B50 | 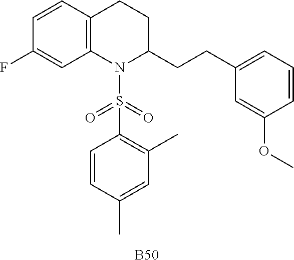 [SMILES: "B50" ; COc1cccc(CCC2CCc3ccc(F)cc3N2S(=O)(=O)c2ccc(C)cc2C)c1] |
| B51 | 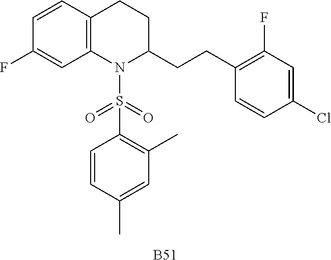 |
| B52 | 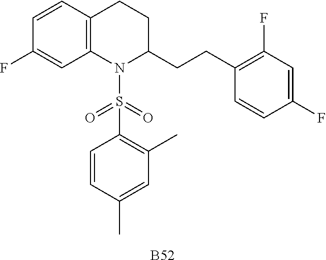 |
| B53 | 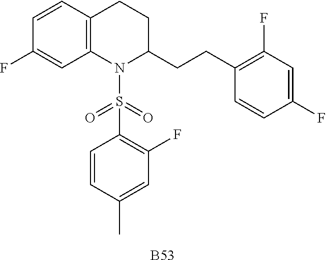 [SMILES: "B53" ; Cc1ccc(S(=O)(=O)N2c3cc(F)ccc3CCC2CCc2ccc(F)cc2F)c(F)c1] |
| B54 | 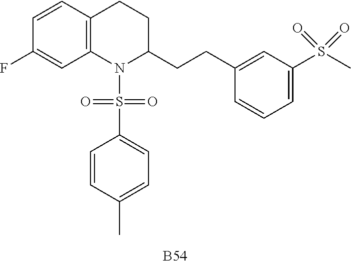 |
| B57 | 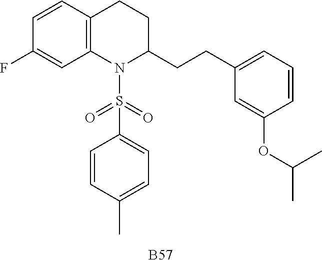 |
| B59 | 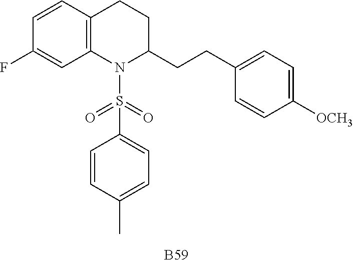 |
| B60 | 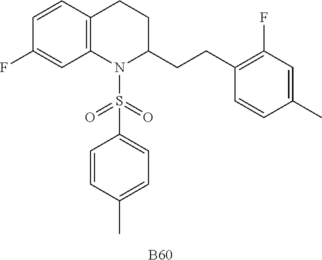 |
| B61 | 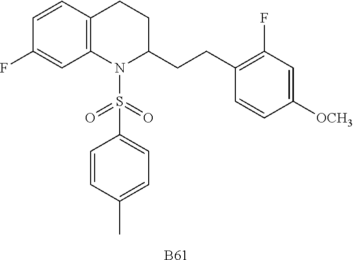 |
| B62 | 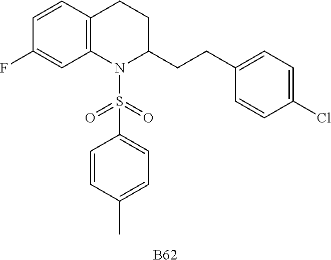 |
| B63 | 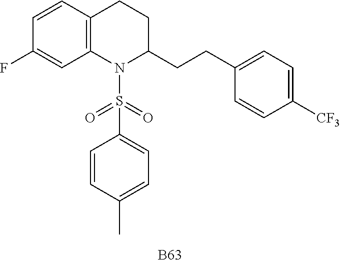 |
| B64 | 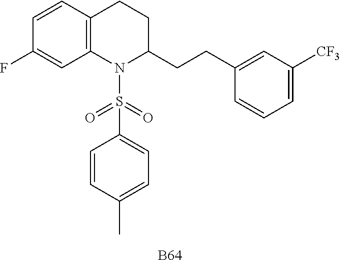 |
| B65 | 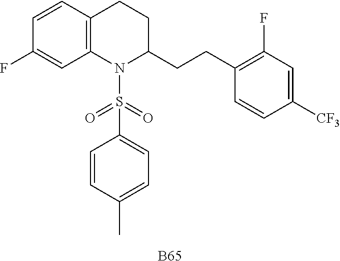 |
| B66 | 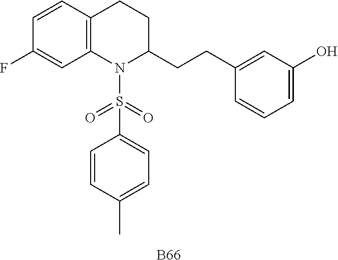 |
| B68 | 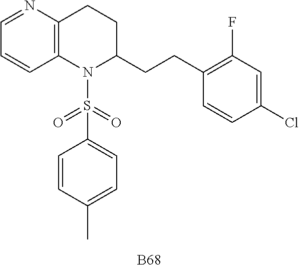 [SMILES: "B68" ; Cc1ccc(S(=O)(=O)N2c3cccnc3CCC2CCc2ccc(Cl)cc2F)cc1] |
| B69 | 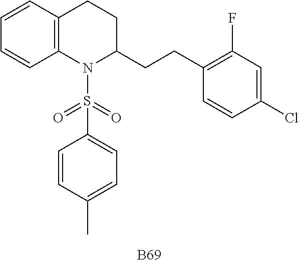 [SMILES: "B69" ; Cc1ccc(S(=O)(=O)N2c3ccccc3CCC2CCc2ccc(Cl)cc2F)cc1] |
| B70 | 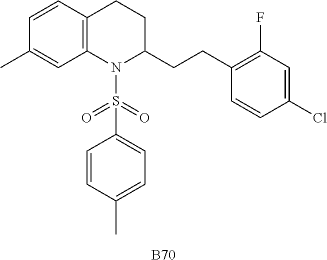 [SMILES: "B70" ; Cc1ccc(S(=O)(=O)N2c3cc(C)ccc3CCC2CCc2ccc(Cl)cc2F)cc1] |
| | 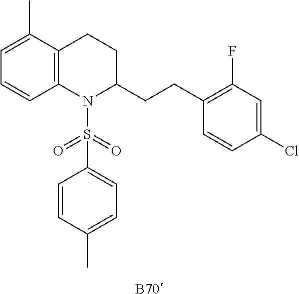 |
| B71 | 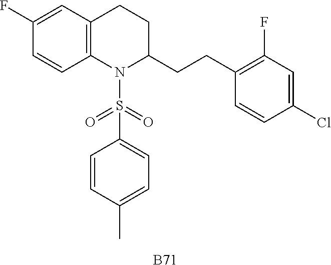 [SMILES: "B71" ; Cc1ccc(S(=O)(=O)N2c3ccc(F)cc3CCC2CCc2ccc(Cl)cc2F)cc1] |
| B72 | 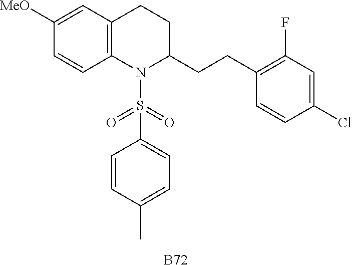 [SMILES: "B72" ; COc1ccc2c(c1)CCC(CCc1ccc(Cl)cc1F)N2S(=O)(=O)c1ccc(C)cc1] |
| B75 | 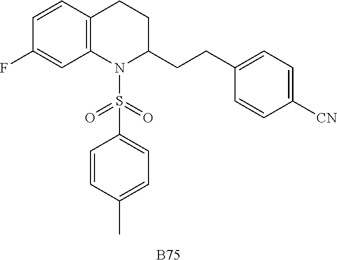 |
| B76 | 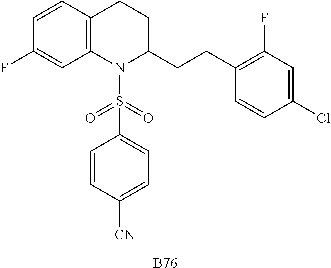 [SMILES: "B76" ; N#Cc1ccc(S(=O)(=O)N2c3cc(F)ccc3CCC2CCc2ccc(Cl)cc2F)cc1] |
| B78 | 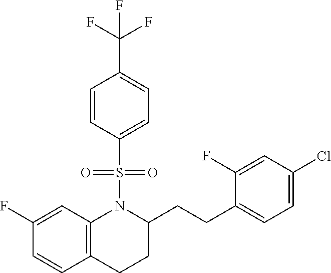 [SMILES: O=S(=O)(c1ccc(C(F)(F)F)cc1)N1c2cc(F)ccc2CCC1CCc1ccc(Cl)cc1F] |
| B79 | 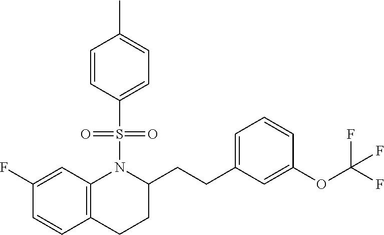 [SMILES: Cc1ccc(S(=O)(=O)N2c3cc(F)ccc3CCC2CCc2cccc(OC(F)(F)F)c2)cc1] |
| B80 | 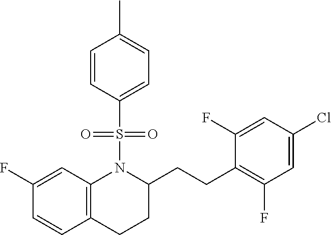 [SMILES: Cc1ccc(S(=O)(=O)N2c3cc(F)ccc3CCC2CCc2c(F)cc(Cl)cc2F)cc1] |
| B81 | 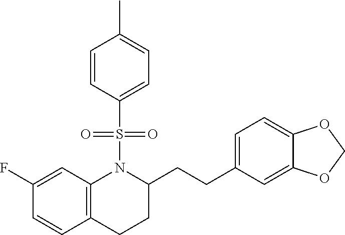 [SMILES: Cc1ccc(S(=O)(=O)N2c3cc(F)ccc3CCC2CCc2ccc3c(c2)OCO3)cc1] |
| B82 | 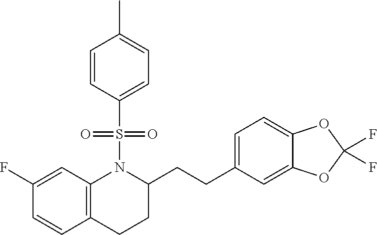 [SMILES: Cc1ccc(S(=O)(=O)N2c3cc(F)ccc3CCC2CCc2ccc3c(c2)OC(F)(F)O3)cc1] |
| B83 | 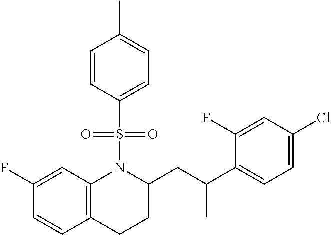 [SMILES: Cc1ccc(S(=O)(=O)N2c3cc(F)ccc3CCC2CC(C)c2ccc(Cl)cc2F)cc1] |
| B84 | 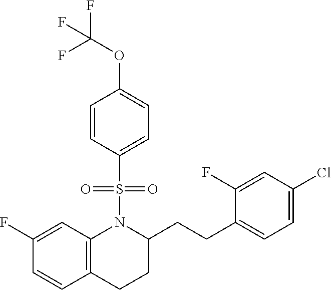 [SMILES: O=S(=O)(c1ccc(OC(F)(F)F)cc1)N1c2cc(F)ccc2CCC1CCc1ccc(Cl)cc1F] |
| B85 | 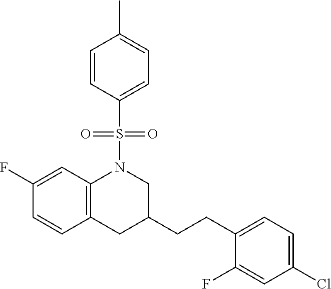 [SMILES: Cc1ccc(S(=O)(=O)N2CC(CCc3ccc(Cl)cc3F)Cc3ccc(F)cc32)cc1] |
| B86 | 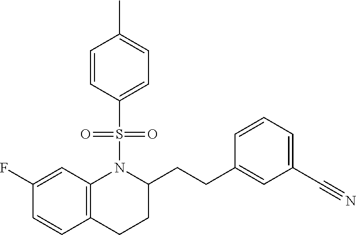 [SMILES: Cc1ccc(S(=O)(=O)N2c3cc(F)ccc3CCC2CCc2cccc(C#N)c2)cc1] |
| B87 | 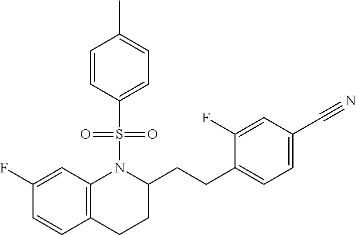 [SMILES: Cc1ccc(S(=O)(=O)N2c3cc(F)ccc3CCC2CCc2ccc(C#N)cc2F)cc1] |
| B88 | 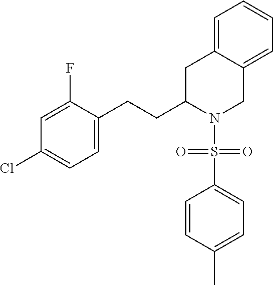 [SMILES: Cc1ccc(S(=O)(=O)N2Cc3ccccc3CC2CCc2ccc(Cl)cc2F)cc1] |
| B90 | 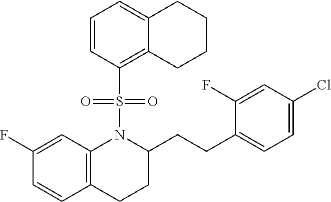 [SMILES: O=S(=O)(c1cccc2c1CCCC2)N1c2cc(F)ccc2CCC1CCc1ccc(Cl)cc1F] |
| B91 | 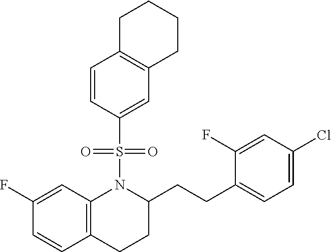 [SMILES: O=S(=O)(c1ccc2c(c1)CCCC2)N1c2cc(F)ccc2CCC1CCc1ccc(Cl)cc1F] |

Exemplary compounds of formula (II) are shown in table 3:

TABLE 3

| | |
|---|---|
| B5 | 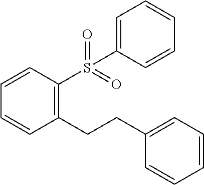 [SMILES: O=S(=O)(c1ccccc1)c1ccccc1CCc1ccccc1] |
| B13 | 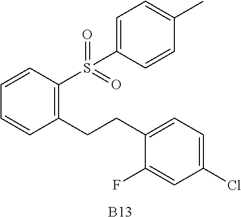 |
| B14 | 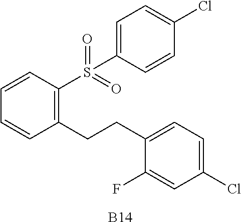 |
| B25 | 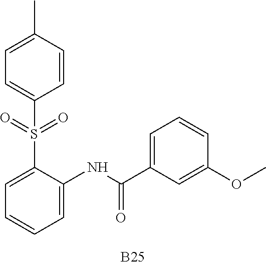 |
| B26 | 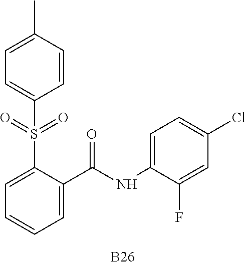 |
| B27 | 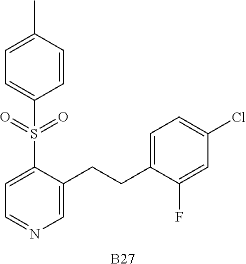 |
| B89 | 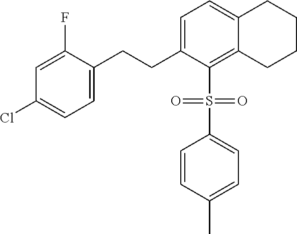 [SMILES: Cc1ccc(S(=O)(=O)c2c(CCc3ccc(Cl)cc3F)ccc3c2CCCC3)cc1] |

Exemplary compounds of formula (IIA) are shown in table 4:

TABLE 4

| | |
|---|---|
| B15 | 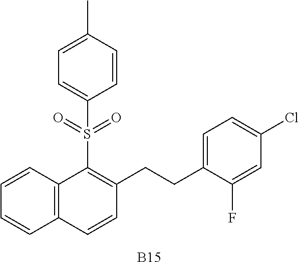 |
| B16 | 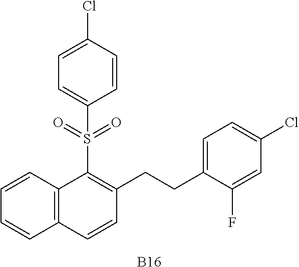 |
| B17 | 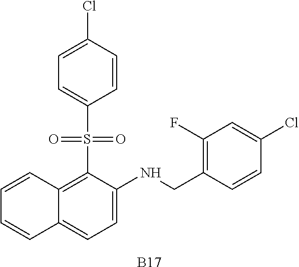 |
| B17′ | 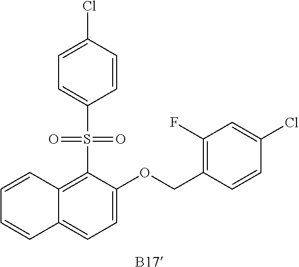 |
| B18 | 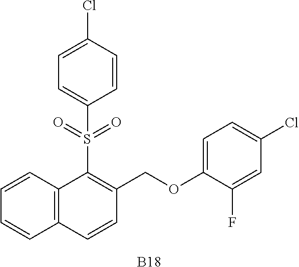 |
| B18′ | 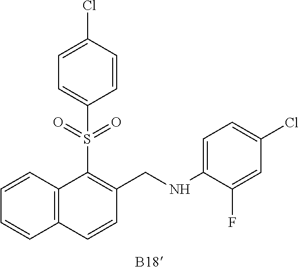 |
| B77 | 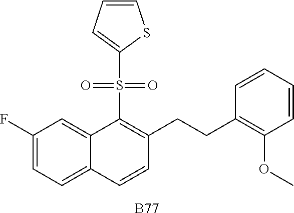 [SMILES: "B77" ; COc1ccccc1CCc1ccc2ccc(F)cc2c1S(=O)(=O)c1cccs1] |

Procedure 1 (B1, B2, B3, B6, B9, B10, B19, B20, B21):

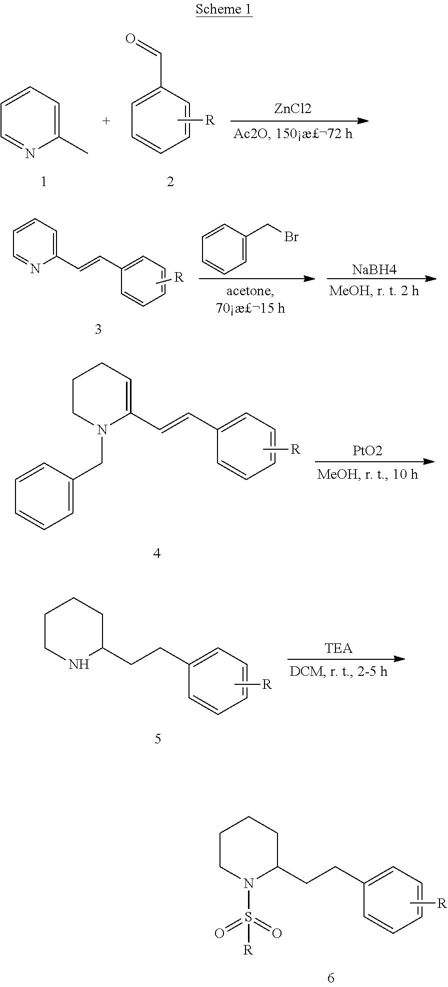

Step 1: To a mixture of 2-methylpyridine (1 equiv.) and substituent benzaldehyde (1 equiv.) in acetic anhydride was added zinc chloride (0.1 equiv.) at room temperature. The mixture was refluxed over 72 h (150° C.). Upon completion by TLC, the reaction mixture was then diluted with saturated sodium bicarbonate solution. The mixture was extracted with ethyl acetate, and the combined organic layers were dried over anhydrous sodium sulfate. The filtrate was concentrated under vacuum and used for next step without purification.

Step 2: To a solution of compound 3 (1 equiv.) in acetone was added benzyl bromide (1.1 equiv.) at room temperature. The mixture was refluxed over 15 h (70° C.). After cooling to 20° C., the precipitated solid was filtered and washed with dichloromethane. The solid was diluted with methanol. Sodium borohydride (2.0 equiv.) was added to the mixture at 0° C. portion wise. After the reaction, the mixture was concentrated under vacuum and then diluted with water. The mixture was extracted with ethyl acetate, and the combined organic layers were washed with brine and dried over anhydrous sodium sulfate. The filtrate was concentrated in vacuum and purified by silica gel column chromatography to afford compound 4 as viscous oil.

Step 3: A mixture of compound 4 and PtO₂ in methanol was stirred at room temperature under atmospheric pressure of hydrogen for 10 hours. The catalyst and solvent were removed and then the residue was purified by silica gel column chromatography to give compound 5 as viscous oil.

Step 4: To a solution of compound 5 and triethylamine in dichloromethane was slowly added substituent aryl sulfonyl chloride at 0° C. The reaction mixture was warmed to room temperature and stirred for 2-5 hours. Water was added to the mixture, and the resulting mixture was extracted three times with dichloromethane.

The obtained organic layer was washed with brine, dried over anhydrous sodium sulfate, filtered, and concentrated under reduced pressure. The residue was purified by silica gel column chromatography to afford compound 6 as off-white to yellow oil.

2-(3-Methoxyphenethyl)-1-(phenylsulfonyl)piperidine (B1-1): 0.61 g of yellow oil (Yield: 10.9%). MS-ESI: [M+1]⁺=360.5. ¹H NMR (300 MHz, CDCl₃): 7.85 (t, 1H), 7.57-7.48 (m, 3H), 7.21 (t, 1H), 6.77-6.74 (m, 3H), 4.13 (m, 1H), 3.87 (m, 1H), 3.82 (s, 3H), 3.07 (t, 1H), 2.63-2.57 (m, 2H), 2.02-1.83 (m, 1H), 1.78-1.72 (m, 1H), 1.56-1.43 (m, 5H), 1.28 (m, 1H).

1-[(4-Fluorophenyl)sulfonyl]-2-(3-methoxyphenethyl)piperidine (B1-2): 0.81 g of yellow oil (Yield: 12.8%). MS-ES: [M+1]⁺=378.5. ¹H NMR (300 MHz, CDCl₃): 7.88-7.83 (m, 2H), 7.24-7.15 (m, 3H), 6.78-6.73 (m, 3H), 4.13 (m, 1H), 3.84-3.79 (m, 1H), 3.82 (s, 3H), 3.06 (t, 1H), 2.62-2.57 (m, 2H), 1.99-1.89 (m, 1H), 1.82-1.73 (m, 1H), 1.57-1.42 (m, 5H), 1.28-1.24 (m, 1H).

2-(3-Methoxyphenethyl)-1-[(4-methoxyphenyl)sulfonyl]piperidine (B1-3): 0.72 g of yellow oil (Yield: 11.1%). MS-ES: [M+1]⁺=390.6. ¹H NMR (300 MHz, CDCl₃): 7.81-7.77 (m, 2H), 7.24-7.18 (m, 1H), 7.00-6.96 (m, 2H), 6.78-6.74 (m, 3H), 4.10 (m, 1H), 3.88 (s, 3H), 3.82 (s, 3H), 3.85-3.79 (m, 1H), 3.05 (t, 1H), 2.64-2.57 (m, 2H), 2.02-1.83 (m, 1H), 1.77-1.72 (m, 1H), 1.53-1.45 (m, 5H), 1.28 (m, 1H).

2-(3-Methoxyphenethyl)-1-tosylpiperidine (B1-4): 0.67 g of yellow oil (Yield: 11.4%). MS-ESI: [M+1]⁺=374.5. ¹H NMR (300 MHz, CDCl₃): 7.76-7.73 (m, 2H), 7.31-7.24 (m, 2H), 7.22-7.19 (m, 1H), 6.78-6.74 (m, 3H), 4.12 (m, 1H), 3.88 (s, 3H), 3.87-3.83 (m, 1H), 3.04 (t, 1H), 2.64-2.58 (m, 2H), 2.44 (s, 3H), 2.02-1.83 (m, 1H), 1.77-1.74 (m, 1H), 1.52-1.46 (m, 5H), 1.28 (m, 1H).

2-(2-Methoxyphenethyl)-1-(phenylsulfonyl)piperidine (B2-1): 0.6 g of yellow oil (Yield: 2.3%). MS-ESI [M+1]⁺=360.4. ¹H NMR (300 MHz, CDCl₃): 7.87-7.84 (m, 2H), 7.55-7.46 (m, 3H), 7.23-7.10 (m, 2H), 6.92-6.84 (m, 2H), 4.13 (m, 1H), 3.87-3.82 (m, 1H), 3.82 (s, 3H), 3.07 (t, 1H), 2.63-2.55 (m, 2H), 1.85-1.72 (m, 2H), 1.56-1.47 (m, 5H), 1.29 (m, 1H).

1-[(4-Fluorophenyl)sulfonyl]-2-(2-methoxyphenethyl)piperidine (B2-2): 0.75 g of yellow oil (Yield: 2.6%). MS-ES: [M+1]⁺=378.5. ¹H NMR (300 MHz, CDCl₃): 7.87-7.82 (m, 2H), 7.23-7.10 (m, 4H), 6.92-6.84 (m, 2H), 4.11 (m, 1H), 3.85-3.80 (m, 1H), 3.82 (s, 3H), 3.07 (t, 1H), 2.61-2.54 (m, 2H), 1.87-1.74 (m, 2H), 1.63-1.49 (m, 5H), 1.31 (m, 1H).

2-(2-Methoxyphenethyl)-1-[(4-methoxyphenyl)sulfonyl]piperidine (B2-3): 0.65 g of yellow oil (Yield: 2.4%). MS-ES: [M+1]⁺=390.5. ¹H NMR (300 MHz, CDCl₃): 7.80-7.77 (m, 2H), 7.22-7.11 (m, 2H), 6.97-6.84 (m, 4H), 4.11 (m, 1H), 3.88 (s, 3H), 3.83 (s, 3H), 3.87-3.78 (m, 1H), 3.05 (t, 1H), 2.64-2.55 (m, 2H), 1.74-1.59 (m, 2H), 1.55-1.46 (m, 5H), 1.31 (m, 1H).

2-(2-Methoxyphenethyl)-1-tosylpiperidine (B2-4): 0.14 g of yellow oil (Yield: 1.4%). MS-ES: [M+1]⁺=374.4. ¹H NMR (300 MHz, CDCl₃): 7.75-7.72 (m, 2H), 7.29-7.27 (m, 3H), 7.22-7.10 (m, 2H), 6.91-6.84 (m, 2H), 4.11 (m, 1H), 3.85-3.79 (m, 1H), 3.82 (s, 3H), 3.07 (t, 1H), 2.63-2.55 (m, 2H), 2.43 (s, 3H), 1.90-1.66 (m, 2H), 1.61-1.46 (m, 5H), 1.31 (m, 1H).

2-Phenethyl-1-(phenylsulfonyl)piperidine (B3-1): 0.88 g of off-white powder (Yield: 1.7%). MS-ESI: [M+1]⁺=330.5. ¹H NMR (300 MHz, CDCl₃): 7.88-7.85 (m, 2H), 7.57-7.48 (m, 3H), 7.32-7.28 (m, 2H), 7.23-7.16 (m, 2H), 4.14 (m, 1H), 3.89-3.83 (m, 1H), 3.06 (t, 1H), 2.66-2.59 (m, 2H), 1.97-1.92 (m, 1H), 1.78-1.73 (m, 1H), 1.55-1.44 (m, 5H), 1.26 (m, 1H).

1-[(4-Fluorophenyl)sulfonyl]-2-phenethylpiperidine (B3-2): 1.2 g of off-white powder (Yield: 2.6%). MS-ES: [M+1]⁺=348.5. ¹H NMR (300 MHz, CDCl₃): 7.89-7.84 (m, 2H), 7.33-7.28 (m, 2H), 7.24-7.15 (m, 5H), 4.11 (m, 1H), 3.86-3.80 (m, 1H), 3.02 (t, 1H), 2.65-2.60 (m, 2H), 1.99-1.92 (m, 1H), 1.83-1.76 (m, 1H), 1.60-1.44 (m, 5H), 1.26 (m, 1H).

1-[(4-Methoxyphenyl)sulfonyl]-2-phenethylpiperidine (B3-3): 0.9 g of off-white powder (Yield: 1.3%). MS-ES: [M+1]⁺=360.4. ¹H NMR (300 MHz, CDCl₃): 7.81-7.77 (m, 2H), 7.32-7.28 (m, 2H), 7.23-7.17 (m, 3H), 6.99-6.96 (m, 2H), 4.10 (m, 1H), 3.89 (s, 3H), 3.85-3.79 (m, 1H), 3.06 (t, 1H), 2.66-2.59 (m, 2H), 1.98-1.94 (m, 1H), 1.78-1.72 (m, 1H), 1.50-1.46 (m, 5H), 1.26 (m, 1H).

2-Phenethyl-1-tosylpiperidine (B3-4): 1.09 g of off-white powder (Yield: 2.6%). MS-ESI: [M+1]⁺=344.4. ¹H NMR (300 MHz, CDCl₃): 7.75-7.73 (m, 2H), 7.31-7.28 (m, 5H), 7.23-7.16 (m, 3H), 4.10 (m, 1H), 3.86-3.80 (m, 1H), 3.06 (t, 1H), 2.66-2.59 (m, 2H), 2.44 (s, 3H), 1.98-1.90 (m, 1H), 1.77-1.69 (m, 1H), 1.57-1.46 (m, 5H), 1.27 (m, 1H).

2-Phenethyl-1-[(2-thienyl)sulfonyl]piperidine (B6-1): 1 g of off-white powder (Yield: 2.6%). MS-ESI: [M+1]⁺=336.3. ¹H NMR (300 MHz, CDCl₃): 7.63-7.51 (m, 2H), 7.33-7.26 (m, 2H), 7.25-7.15 (m, 3H), 7.12-7.05 (m, 1H), 4.27-4.18 (m, 1H), 3.95-3.82 (m, 1H), 3.17-3.01 (m, 1H), 2.73-2.57 (m, 2H), 2.06-1.89 (m, 1H), 1.83-1.70 (m, 1H), 1.60-1.47 (m, 5H), 1.41-1.27 (m, 1H).

2-(2-Methoxyphenethyl)-1-[(2-thienyl)sulfonyl]piperidine (B6-2): 0.65 g of yellow oil (Yield: 1.7%). MS-ES: [M+1]⁺=366.5. ¹H NMR (300 MHz, CDCl₃): 7.59-7.51 (m, 2H), 7.25-7.03 (m, 3H), 6.94-6.82 (m, 2H), 4.24-4.12 (m, 1H), 3.94-3.87 (m, 1H), 3.83 (s, 3H), 3.17-3.03 (m, 1H), 2.70-2.58 (m, 2H), 1.97-1.69 (m, 2H), 1.62-1.47 (m, 5H), 1.43-1.30 (m, 1H).

3-[2-[1-(Phenylsulfonyl)-2-piperidyl]ethyl]pyridine (B9-1): 0.09 g of yellow powder (Yield: 1.5%). MS-ESI: [M+1]⁺=331.5. ¹H NMR (300 MHz, CDCl₃): 8.49 (d, 1H), 7.99-7.87 (m, 2H), 7.57-7.41 (m, 3H), 7.07 (d, 1H), 6.77 (s, 1H), 6.05-5.90 (m, 1H), 3.51-3.39 (m, 1H), 3.30-3.01 (m, 2H), 2.34-2.16 (m, 2H), 2.08-1.98 (m, 1H), 1.95-1.86 (m, 1H), 1.84-1.66 (m, 3H), 1.57-1.24 (m, 3H).

3-[2-[1-[(4-Fluorophenyl)sulfonyl]-2-piperidyl]ethyl]pyridine (B9-2): 0.14 g of yellow powder (Yield: 2.3%). MS-ES: [M+1]⁺=349.5. ¹H NMR (300 MHz, CDCl₃): 8.45 (d, 1H), 8.01-7.87 (m, 2H), 7.23-7.14 (m, 3H), 6.81 (s, 1H), 6.05-5.88 (m, 1H), 3.54-3.41 (m, 1H), 3.32-3.11 (m, 2H), 2.35-2.16 (m, 2H), 2.01-1.98 (m, 1H), 1.96-1.66 (m, 4H), 1.58-1.25 (m, 3H).

3-[2-[1-[(4-Methoxyphenyl)sulfonyl]-2-piperidyl]ethyl]pyridine (B9-3): 0.07 g of yellow powder (Yield: 1.2%). MS-ESI: [M+1]⁺=361.5. ¹H NMR (300 MHz, CDCl₃): 8.46 (d, 1H), 7.84 (d, 2H), 7.03 (d, 1H), 6.93 (d, 2H), 6.71 (s, 1H), 5.99-5.90 (m, 1H), 3.84 (s, 3H), 3.46-3.37 (m, 1H), 3.25-3.07 (m, 2H), 2.30-2.16 (m, 2H), 2.08-1.97 (m, 1H), 1.94-1.86 (m, 1H), 1.82-1.64 (m, 3H), 1.52-1.27 (m, 3H).

3-[2-(1-Tosyl-2-piperidyl)ethyl]pyridine (B9-4): 0.16 g of brown powder (Yield: 2.2%). MS-ESI: [M+1]⁺=345.5. ¹H NMR (300 MHz, CDCl₃): 8.47 (d, 1H), 7.81 (d, 2H), 7.26 (d, 2H), 7.05 (d, 1H), 6.75 (s, 1H), 6.02-5.90 (m, 1H), 3.50-3.39 (m, 1H), 3.28-3.08 (m, 2H), 2.40 (s, 3H), 2.31-2.16 (m, 2H), 2.08-1.97 (m, 1H), 1.94-1.86 (m, 1H), 1.82-1.65 (m, 3H), 1.57-1.29 (m, 3H).

2-(4-Methylphenethyl)-1-(phenylsulfonyl)piperidine (B10-1): 1.1 g of yellow oil (Yield: 3.5%). MS-ESI: [M+1]⁺=344.5. ¹H NMR (300 MHz, CDCl₃): 7.94-7.82 (m, 2H), 7.61-7.47 (m, 3H), 7.16-7.01 (m, 4H), 4.21-4.08 (m, 1H), 3.92-3.79 (m, 1H), 3.14-3.99 (m, 1H), 2.66-2.52 (m, 2H), 2.34 (s, 3H), 2.00-1.87 (m, 1H), 1.79-1.66 (m, 1H), 1.58-1.38 (m, 5H), 1.33-1.14 (m, 1H).

1-[(4-Fluorophenyl)sulfonyl]-2-(4-methylphenethyl)piperidine (B10-2): 1.2 g of off-white solid (Yield: 3.6%). MS-ESI: [M+1]⁺=362.5. ¹H NMR (300 MHz, CDCl₃): 7.91-7.80 (m, 2H), 7.24-7.02 (m, 6H), 4.17-4.05 (m, 1H), 3.90-3.76 (m, 1H), 3.13-2.97 (m, 1H), 2.65-2.50 (m, 2H), 2.34 (s, 3H), 2.00-1.85 (m, 1H), 1.81-1.69 (m, 1H), 1.58-1.40 (m, 5H), 1.33-1.15 (m, 1H).

1-[(4-Methoxyphenyl)sulfonyl]-2-(4-methylphenethyl)piperidine (B10-3): 1.3 g of off-white solid (Yield: 3.7%). MS-ESI: [M+1]⁺=374.6. ¹H NMR (300 MHz, CDCl₃): 7.78 (d, 2H), 7.16-7.02 (m, 4H), 6.97 (d, 2H), 4.16-4.04 (m, 1H), 3.88 (s, 3H), 3.86-3.75 (m, 1H), 3.10-2.96 (m, 1H), 2.67-2.50 (m, 2H), 2.34 (s, 3H), 2.00-1.86 (m, 1H), 1.79-1.66 (m, 1H), 1.57-1.40 (m, 5H), 1.35-1.14 (m, 1H).

2-(4-Methylphenethyl)-1-tosylpiperidine (B10-4): 1.3 g of off-white solid (Yield: 3.7%). MS-ESI: [M+1]⁺=358.6. ¹H NMR (300 MHz, CDCl₃): 7.73 (d, 2H), 7.30 (d, 2H), 7.15-7.01 (m, 4H), 4.17-4.06 (m, 1H), 3.89-3.77 (m, 1H), 3.11-2.97 (m, 1H), 2.67-2.50 (m, 2H), 2.44 (s, 3H), 2.34 (s, 3H), 2.00-1.86 (m, 1H), 1.79-1.66 (m, 1H), 1.57-1.40 (m, 5H), 1.35-1.14 (m, 1H).

2-(4-Chloro-2-fluorophenethyl)-1-(phenylsulfonyl)piperidine (B10-5): 0.12 g of white solid (Yield: 0.9%). MS-ES: [M+1]⁺=382.4. ¹H NMR (300 MHz, CDCl₃): 7.86 (d, 2H), 7.60-7.49 (m, 3H), 7.17-7.03 (m, 3H), 4.12 (m, 1H), 3.88-3.82 (m, 1H), 3.06 (t, 1H), 2.67-2.59 (m, 2H), 1.99-1.90 (m, 1H), 1.75-1.66 (m, 1H), 1.56-1.43 (m, 5H), 1.28-1.17 (m, 1H).

2-(4-Chloro-2-fluorophenethyl)-1-[(4-fluorophenyl)sulfonyl]piperidine (B10-6): 0.16 g of colorless oil (Yield: 0.4%). MS-ES: [M+1]⁺=400.3. ¹H NMR (300 MHz, CDCl₃): 7.88-7.84 (m, 2H), 7.22-7.03 (m, 5H), 4.12-4.08 (m, 1H), 3.86-3.80 (m, 1H), 3.06 (t, 1H), 2.67-2.58 (m, 2H), 1.96-1.92 (m, 1H), 1.74-1.65 (m, 1H), 1.57-1.43 (m, 5H), 1.27-1.20 (m, 1H).

2-(4-Chloro-2-fluorophenethyl)-1-[(4-methoxyphenyl)sulfonyl]piperidine (B10-7): 0.65 g of colorless oil (Yield: 1.6%). MS-ES: [M+1]⁺=412.5. ¹H NMR (300 MHz, CDCl₃): 7.78 (d, 2H), 7.18-6.95 (m, 5H), 4.08 (m, 1H), 3.88 (s, 3H), 3.85-3.79 (m, 1H), 3.04 (t, 1H), 2.68-2.58 (m, 2H), 2.02-1.90 (m, 1H), 1.71-1.65 (m, 1H), 1.51-1.44 (m, 5H), 1.27 (m, 1H).

2-(4-Chloro-2-fluorophenethyl)-1-tosylpiperidine (B10-8): 0.5 g of colorless oil (Yield: 1.3%). MS-ESI: [M+1]⁺=396.5. ¹H NMR (300 MHz, CDCl₃): 7.73 (d, 2H), 7.31-7.28 (m, 2H), 7.17-7.02 (m, 3H), 4.10 (m, 1H), 3.86-3.76 (m, 1H), 3.04 (t, 1H), 2.68-2.59 (m, 2H), 2.44 (s, 3H), 1.96-1.91 (m, 1H), 1.71-1.63 (m, 1H), 1.51-1.44 (m, 5H), 1.27-1.21 (m, 1H).

5-[[2-(4-Chloro-2-fluorophenethyl)-1-piperidinyl]sulfonyl]-2-methylpyridine (B19): 0.52 g of colorless oil (Yield: 1.3%). MS-ES: [M+1]⁺=397.4. ¹H NMR (300 MHz, CDCl₃): 8.94 (s, 1H), 8.00 (d, 2H), 7.31-7.28 (m, 1H), 7.09-7.03 (m, 3H), 4.12 (m, 1H), 3.86-3.80 (m, 1H), 3.08 (t, 1H), 2.66 (s, 3H), 2.66-2.59 (m, 2H), 1.98-1.93 (m, 1H), 1.75-1.70 (m, 1H), 1.54-1.43 (m, 5H), 1.30-1.21 (m, 1H).

2-(3-Chloro-4-fluorophenethyl)-1-tosylpiperidine (B20): 0.3 g of yellow oil (Yield: 1.2%). MS-ESI: [M+1]⁺=396.4. ¹H NMR (300 MHz, CDCl₃): 7.73 (d, 2H), 7.31 (d, 2H), 7.19 (d, 1H), 7.05 (d, 2H), 4.17-4.06 (m, 1H), 3.89-3.78 (m, 1H), 3.11-2.97 (m, 1H), 2.69-2.53 (m, 2H), 2.45 (s, 3H), 2.04-1.87 (m, 1H), 1.74-1.64 (m, 1H), 1.56-1.40 (m, 5H), 1.32-1.10 (m, 1H).

2-(3-Chlorophenethyl)-1-tosylpiperidine (B21): 1.9 g of yellow oil (Yield: 1.4%). MS-ESI: [M+1]⁺=378.4. ¹H NMR (300 MHz, CDCl₃): 7.74 (d, 2H), 7.31 (d, 2H), 7.25-7.15 (m, 3H), 7.07 (d, 1H), 4.17-4.06 (m, 1H), 3.89-3.78 (m, 1H), 3.11-2.97 (m, 1H), 2.69-2.53 (m, 2H), 2.45 (s, 3H), 2.04-1.87 (m, 1H), 1.74-1.64 (m, 1H), 1.56-1.40 (m, 5H), 1.32-1.10 (m, 1H).

Procedure 2 (B7, B11, B12, B28, B29, B30, B32, B33, B34, B40, B41, B44, B45, B46, B61, B62, B63, B64, B65, B66, B68, B69, B70, B70′, B71, B72, B74, B75, B76)

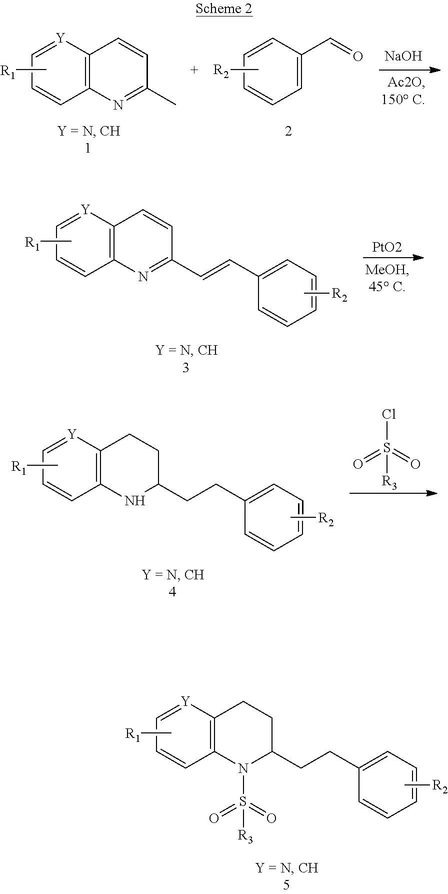

Step 1: To a stirred solution of 7-fluoro-2-methylquinoline (1 equiv.) in acetic anhydride at room temperature was added substituent benzaldehyde (2.8 equiv.) and sodium hydroxide (0.2 equiv.). The reaction mixture was stirred at a refluxed temperature (150° C.) for 48 hours. After the mixture was cooled down to room temperature, water and dichloromethane was added to the reaction mixture. Then, the mixture was stirred for 3 hours. The organic layer was separated and washed with sodium hydroxide solution (4M) until it became slightly basic. This reaction mixture was then extracted with dichloromethane three times. The combined organic layer was washed with brine, dried over anhydrous sodium sulfate, filtered, and concentrated under reduced pressure. The residue was diluted with petroleum and the precipitated brown solid was filtered to give compound 3.

Step 2: A mixture of compound 3 and PtO₂ in methanol was stirred at room temperature under atmospheric pressure of hydrogen for 15 hours. The catalyst and solvent were removed and then the residue was purified by silica gel column chromatography to give compound 5 as viscous oil.

Step 3: To a solution of compound 4 and triethylamine in dichloromethane was slowly added substituent aryl sulfonyl chloride at 0° C. The reaction mixture was warmed to room temperature and stirred for 2 hours. Water was added to the mixture, and the resulting mixture was extracted three times with dichloromethane. The obtained organic layer was washed with brine, dried over anhydrous sodium sulfate, filtered, and concentrated under reduced pressure. The residue was purified by silica gel column chromatography to afford compound 5 as off-white to yellow solid.

7-Fluoro-2-(2-methoxyphenethyl)-1-[(2-thienyl)sulfonyl]-1,2,3,4-tetrahydroquinoline (B7-4): 2.1 g of yellow oil (Yield: 10.9%). MS-ES: [M+H]⁺=432.5. ¹H NMR (300 MHz, CDCl₃): 7.59 (q, 1H), 7.53 (q, 2H), 7.33 (m, 1H), 7.19-7.13 (m, 2H), 7.04-6.97 (m, 2H), 6.91-6.81 (m, 2H), 4.49-4.45 (m, 1H), 3.76 (s, 3H), 2.79-2.69 (m, 2H), 2.55 (m, 1H), 2.10 (m, 1H), 1.87-1.70 (m, 3H), 1.59-1.50 (m, 1H).

2-(4-Chloro-2-fluorophenethyl)-7-fluoro-1-tosyl-1,2,3,4-tetrahydroquinoline (B11): 1.1 g of yellow oil (Yield: 17%). MS-ES: [M+H]⁺=461.9. ¹H NMR (300 MHz, CDCl₃): 7.62-7.58 (m, 1H), 7.43 (d, 2H), 7.21-7.15 (m, 2H), 7.08-6.85 (m, 5H), 4.38-4.34 (m, 1H), 2.78-2.72 (m, 2H), 2.46-2.40 (m, 1H), 2.39 (s, 3H), 2.02-1.97 (m, 1H), 1.83-1.63 (m, 3H), 1.49-1.45 (m, 1H).

2-(4-Chloro-2-fluorophenethyl)-1-[(4-chlorophenyl)sulfonyl]-7-fluoro-1,2,3,4-tetrahydroquinoline (B12): 1.0 g of yellow oil (Yield: 14%). MS-ES: [M+H]⁺=481.8. ¹H NMR (300 MHz, CDCl₃): 7.61-7.57 (m, 1H), 7.49-7.36 (d, 4H), 7.20-6.88 (m, 5H), 4.39-4.34 (m, 1H), 2.77-2.73 (m, 2H), 2.49-2.47 (m, 1H), 2.03-1.97 (m, 1H), 1.84-1.66 (m, 3H), 1.53-1.48 (m, 1H).

2-(4-Chloro-2-fluorophenethyl)-7-fluoro-1-[(4-isopropylphenyl)sulfonyl]-1,2,3,4-tetrahydroquinoline (B28): 0.8 g of off-white solid (Yield: 12%). MS-ES: [M+H]⁺=490.5. ¹H NMR (300 MHz, CDCl₃): 7.63-7.59 (m, 1H), 7.46 (d, 2H), 7.25 (d, 2H), 7.20-6.86 (m, 5H), 4.38-4.34 (m, 1H), 2.97-2.93 (m, 1H), 2.78-2.72 (m, 2H), 2.47-2.44 (m, 1H), 2.00-1.95 (m, 1H), 1.80-1.61 (m, 3H), 1.57-1.45 (m, 1H), 1.25 (d, 6H).

2-(4-Chloro-2-fluorophenethyl)-7-fluoro-1-(m-tolylsulfonyl)-1,2,3,4-tetrahydroquinoline (B29): 0.8 g of white solid (Yield: 12%). MS-ES: [M+H]⁺=462.5. ¹H NMR (300 MHz, CDCl₃): 7.63-7.58 (m, 1H), 7.35-7.28 (m, 4H), 7.21-7.16 (m, 1H), 7.09-6.86 (m, 4H), 4.38-4.33 (m, 1H), 2.76-2.72 (m, 2H), 2.46-2.43 (m, 1H), 2.34 (s, 3H), 2.00-1.93 (m, 1H), 1.84-1.63 (m, 3H), 1.49-1.45 (m, 1H).

2-(4-Chloro-2-fluorophenethyl)-7-fluoro-1-(o-tolylsulfonyl)-1,2,3,4-tetrahydroquinoline (B30): 0.45 g of white solid (Yield: 10.4%). MS-ES: [M+H]⁺=462.4. ¹H NMR (300 MHz, CDCl₃): 7.90-7.87 (m, 1H), 7.52-7.42 (m, 2H), 7.32-7.12 (m, 3H), 7.07-6.83 (m, 5H), 4.25-4.21 (m, 1H), 2.75-2.71 (m, 2H), 2.59-2.51 (m, 1H), 2.34-2.23 (m, 1H), 2.26 (s, 1H), 1.84-1.73 (m, 1H), 1.71-1.59 (m, 3H), 1.56-1.49 (m, 1H).

2-(4-Chloro-2-fluorophenethyl)-7-fluoro-1-[(6-methyl-3-pyridyl)sulfonyl]-1,2,3,4-tetrahydroquinoline (B32): 0.052 g of yellow solid (Yield: 2.3%). MS-ES: [M+H]⁺=463.5. ¹H NMR (300 MHz, CDCl₃): 8.69 (s, 1H), 7.67-7.58 (m, 2H), 7.26-7.15 (m, 2H), 7.09-6.86 (m, 4H), 4.44-4.40 (m, 1H), 2.82-2.74 (m, 2H), 2.56 (s, 3H), 2.50-2.46 (m, 1H), 2.04-1.93 (m, 1H), 1.81-1.59 (m, 3H), 1.56-1.49 (m, 1H).

2-(4-Chloro-2-fluorophenethyl)-7-fluoro-1-[(5-methyl-2-pyridyl)sulfonyl]-1,2,3,4-tetrahydroquinoline (B33): 0.2 g of yellow solid (Yield: 8.1%). MS-ES: [M+H]⁺=463.5. ¹H NMR (300 MHz, CDCl₃): 8.48 (s, 1H), 7.69-7.67 (m, 1H), 7.61-7.57 (m, 2H), 7.18-6.92 (m, 4H), 6.82-6.75 (m, 1H), 4.73-4.69 (m, 1H), 2.76-2.71 (m, 2H), 2.58-2.52 (m, 1H), 2.41 (s, 3H), 2.33-2.26 (m, 1H), 2.15-2.07 (m, 1H), 1.88-1.59 (m, 4H).

2-(4-Chloro-2-fluorophenethyl)-7-fluoro-1-[(5-methyl-2-thienyl)sulfonyl]-1,2,3,4-tetrahydroquinoline (B34): 0.06 g of yellow solid (Yield: 2.6%). MS-ES: [M+H]⁺=468.5. ¹H NMR (300 MHz, CDCl₃): 7.58-7.54 (d, 1H), 7.25-7.14 (m, 2H), 7.08-6.96 (m, 3H), 6.88-6.85 (m, 1H), 6.68 (t, 1H), 4.47-4.39 (m, 1H), 2.81-2.72 (m, 2H), 2.60-2.49 (m, 1H), 2.51 (s, 3H), 2.33-2.26 (m, 1H), 2.27-2.14 (m, 1H), 1.88-1.59 (m, 4H).

2-(2,4-Difluorophenethyl)-7-fluoro-1-tosyl-1,2,3,4-tetrahydroquinoline (B40): 0.32 g of white solid (Yield: 6.7%). MS-ES: [M+H]⁺=446.6. ¹H NMR (400 MHz, CDCl3) δ 7.59 (dd, 1H), 7.41 (d, 2H), 7.21-7.13 (m, 3H), 6.93 (dd, J=8.4, 6.4 Hz, 1H), 6.87-6.69 (m, 3H), 2.81-2.64 (m, 2H), 2.50-2.40 (m, 1H), 2.38 (s, 3H), 1.98 (dt, 1H), 1.86-1.72 (m, 1H), 1.72-1.51 (m, 3H), 1.51-1.36 (m, 1H).

2-(2-Chloro-4-fluorophenethyl)-7-fluoro-1-tosyl-1,2,3,4-tetrahydroquinoline (B41): 0.99 g of colorless solid (Yield: 20%). MS-ES: [M+H]p=462.5. ¹H NMR (300 MHz, CDCl₃): 7.65-7.61 (d, 1H), 7.47-7.44 (d, 2H), 7.25-7.19 (m, 3H), 7.09-7.05 (m, 1H), 6.99-6.86 (m, 3H), 4.41-4.39 (m, 1H), 2.88-2.81 (m, 2H), 2.53-2.49 (m, 1H), 2.40 (s, 3H), 2.13-1.96 (m, 1H), 1.72-1.63 (m, 3H), 1.57-1.51 (m, 1H).

2-(3-Chloro-4-fluorophenethyl)-7-fluoro-1-tosyl-1,2,3,4-tetrahydroquinoline (B44): 0.10 g of white solid (Yield: 9.8%). MS-ES: [M+H]⁺=461.1. ¹H NMR (400 MHz, CDCl₃) δ 7.62 (dd, 1H), 7.43 (d, 2H), 7.24-7.17 (m, 3H), 7.08-7.03 (m, 2H), 6.96 (dd, 1H), 6.86 (td, 1H), 4.39-4.33 (m, 1H), 2.73 (t, 2H), 2.55-2.42 (m, 1H), 2.40 (s, 3H), 2.00 (dt, 1H), 1.92-1.77 (m, 1H), 1.74-1.60 (m, 2H), 1.50-1.38 (m, 1H).

7-Fluoro-2-(3-methoxyphenethyl)-1-tosyl-1,2,3,4-tetrahydroquinoline (B45): 0.18 g of white solid (Yield: 21.2%). MS-ES: [M+H]⁺=440.5. ¹H NMR (400 MHz, CDCl₃) δ 7.59 (dd, 1H), 7.41 (d, 2H), 7.21-7.13 (m, 3H), 6.95-6.90 (m, 1H), 6.82 (td, 1H), 6.76-6.69 (m, 3H), 4.40-4.32 (m, 1H), 3.79 (s, 3H), 2.75-2.65 (m, 2H), 2.50-2.40 (m, 1H), 2.37 (s, 3H), 2.02-1.92 (m, 1H), 1.92-1.80 (m, 1H), 1.73-1.57 (m, 2H), 1.49-1.39 (m, 1H).

2-(2-Chloro-4-fluorophenethyl)-1-[(2,4-dimethylphenyl)sulfonyl]-7-fluoro-1,2,3,4-tetrahydroquinoline (B46): 0.05 g of viscous oil (Yield: 10.5%). MS-ES: [M+H]⁺=476.7. ¹H NMR (400 MHz, CDCl₃) δ 7.79 (d, 1H), 7.50 (dd, 1H), 7.19 (dd, 1H), 7.10-7.05 (m, 3H), 7.03-6.97 (m, 1H), 6.93-6.82 (m, 2H), 4.39-4.15 (m, 1H), 2.89-2.70 (m, 2H), 2.65-2.52 (m, 1H), 2.37 (s, 3H), 2.24 (s, 3H), 1.84-1.72 (m, 1H), 1.72-1.62 (m, 2H), 1.62-1.51 (m, 2H).

7-Fluoro-2-(2-fluoro-5-methoxyphenethyl)-1-tosyl-1,2,3,4-tetrahydroquinoline (B47) 0.12 g of white solid (Yield: 21.2%). MS-ES: [M+H]⁺=458.6. ¹H NMR (400 MHz, CDCl₃) δ 7.61 (dd, 2.6 Hz, 1H), 7.44 (d, 2H), 7.20 (d, 2H), 6.98-6.82 (m, 3H), 6.78 (dd, 1H), 6.71-6.66 (m, 1H), 4.43-4.35 (m, 1H), 3.80 (s, 3H), 2.82-2.70 (m, 2H), 2.52-2.43 (m, 1H), 2.40 (s, 3H), 2.05-1.97 (m, 1H), 1.90-1.79 (m, 1H), 1.74-1.62 (m, 2H), 1.51-1.44 (m, 1H).

7-Fluoro-2-(4-fluoro-3-methoxyphenethyl)-1-tosyl-1,2,3,4-tetrahydroquinoline (B48) 0.1 g of light yellow solid (Yield: 14.3%). MS-ES: [M+H]⁺:458.7. ¹H NMR (400 MHz, CDCl3) δ 7.63 (dd, 1H), 7.42 (d, 2H), 7.19 (d, 2H), 7.01-6.93 (m, 2H), 6.89-6.83 (m, 2H), 6.70-6.66 (m, 1H), 4.36 (m, 1H). 3.91 (s, 3H), 2.73 (t, 2H), 2.53-2.42 (m, 1H), 2.40 (s, 3H), 2.05-1.83 (m, 2H), 1.72-1.61 (m, 2H), 1.49-1.38 (m, 1H).

1-[(2,4-Dimethylphenyl)sulfonyl]-7-fluoro-2-(2-fluoro-5-methoxyphenethyl)-1,2,3,4-tetrahydroquinoline (B49) 0.058 g of white solid (Yield: 11.1%). MS-ES: [M+H]⁺=472.8. ¹H NMR (400 MHz, CDCl₃) δ 7.77 (d, 1H), 7.48 (dd, 1H), 7.10-6.97 (m, 3H), 6.93-6.81 (m, 2H), 6.74 (dd, 1H), 6.70-6.65 (m, 1H), 4.29-4.20 (m, 1H), 3.79 (s, 3H), 2.73-2.65 (m, 2H), 2.63-2.56 (m, 1H), 2.37 (s, 3H), 2.35-2.27 (m, 1H), 2.22 (s, 3H), 1.89-1.77 (m, 1H), 1.74-1.61 (m, 3H).

1-[(2,4-Dimethylphenyl)sulfonyl]-7-fluoro-2-(3-methoxyphenethyl)-1,2,3,4-tetrahydroquinoline (B50) 0.11 g of white solid (Yield: 32.1%). MS-ES: [M+H]⁺=454.7. ¹H NMR (400 MHz, CDCl₃) δ 7.76 (d, 1H), 7.49 (dd, 1H), 7.19 (t, 1H), 7.07 (d, 1H), 7.04 (s, 1H), 7.02-6.97 (m, 1H), 6.84 (td, 1H), 6.76-6.71 (m, 3H), 4.28-4.18 (m, 1H), 3.81 (s, 3H), 2.75-2.65 (m, 2H), 2.62-2.51 (m, 1H), 2.37 (s, 3H), 2.35-2.26 (m, 1H), 2.22 (s, 3H), 1.93-1.81 (m, 1H), 1.73-1.58 (m, 3H).

2-(4-Chloro-2-fluorophenethyl)-1-[(2,4-dimethylphenyl)sulfonyl]-7-fluoro-1,2,3,4-tetrahydroquinoline (B51) 0.17 g of white solid (Yield: 32.4%). MS-ES: [M+H]⁺=476.7. ¹H NMR (400 MHz, CDCl₃) δ 7.76 (d, 1H), 7.49 (dd, 1H), 7.14 (t, 1H), 7.11-6.97 (m, 5H), 6.85 (td, 1H), 4.26-4.15 (m, 1H), 2.79-2.65 (m, 2H), 2.63-2.50 (m, 1H), 2.37 (s, 3H), 2.30 (dt, 1H), 2.21 (s, 3H), 1.84-1.73 (m, 1H), 1.71-1.62 (m, 2H), 1.56-1.49 (m, 1H).

2-(2,4-Difluorophenethyl)-1-[(2,4-Dimethylphenyl)sulfonyl]-7-fluoro-1,2,3,4-tetrahydroquinoline (B52) 0.3 g of white solid (Yield: 22.9%). MS-ES: [M+H]⁺: 460.6. ¹H NMR (400 MHz, CDCl3)

δ 7.77 (d, 1H), 7.49 (dd, 1H), 7.18-7.12 (m, 1H), 7.10-6.96 (m, 3H), 6.89-6.69 (m, 3H), 4.25-4.19 (m, 1H), 2.74-2.69 (m, 2H), 2.69-2.53 (m, 1H), 2.37 (s, 3H), 2.37-2.27 (dt, 1H), 2.22 (s, 3H), 1.82-1.74 (m, 1H), 1.71-1.60 (m, 2H), 1.53 (m, 1H).

2-(2,4-Difluorophenethyl)-7-fluoro-1-[(2-fluoro-4-methylphenyl)sulfonyl]-1,2,3,4-tetrahydroquinoline (B53)

Prepare 2-fluoro-4-methylbenzenesulfonyl chloride: To a 1000 mL three necked flask was added 90 mL H₂O, to the stirred mixture was dropped SOCl₂ (29.8 g, 219.7 mmol, 5.5 eq) under 0° C.−5° C., then the reaction was stirred at room temperature overnight. CuCl (0.15 g) was added to the reaction mixture (solution A). To a 500 mL three necked flask was added 50 mL HCl, to the stirred mixture was added 2-fluoro-4-methylaniline (5 g, 39.9 mmol, 1.0 eq) under 0° C.-5° C., then the reaction mixture was dropped NaNO₂ (3.59 g, 52 mmol, 1.0 eq) aqueous solution (25 mL) under −5° C.-0° C. Then the reaction mixture was stirred at 0° C. for 1 hour (solution B). Solution B was dropped to solution A under 0° C.-5° C., then the reaction mixture was stirred at room temperature overnight. The reaction mixture was poured onto 500 mL ice water and stirred for 0.5 h, extracted with EA (100 mL*3). The combined organic layers were washed with sat. NaHCO₃ aqueous solution and dried over Na₂SO₄, filtered and concentrated in vacuum. The residue was purified by column chromatography on silica gel to yield the title product (3 g, HPLC: 95%) as a light yellow solid. (Yield: 36.1%).

Prepare B53: B53 was prepared from 2-fluoro-4-methylbenzenesulfonyl chloride according the procedure 2, 0.054 g of light yellow solid was obtained (Yield: 3.1%). MS-ES: [M+H]⁺: 464.4. ¹H NMR (400 MHz, CDCl₃) δ 7.68 (t, J=7.7 Hz, 1H), 7.53 (dd, J=10.9, 2.5 Hz, 1H), 7.19-7.13 (m, 1H), 7.04-6.90 (m, 3H), 6.85-6.71 (m, 3H), 4.59-4.48 (m, 1H), 2.74-2.66 (m, 2H), 2.68-2.57 (m, 1H), 2.46-2.34 (m, 4H), 2.02-1.94 (m, 1H), 1.91-1.78 (m, 1H), 1.77-1.60 (m, 2H).

7-Fluoro-2-(3-isopropoxyphenethyl)-1-tosyl-1,2,3,4-tetrahydroquinoline (B57) Prepare 3-isopropoxy-benzaldehyde: To a 500 mL three necked flask was added 200 mL DMF, to the stirred mixture was added 3-hydroxybenzaldehyde (5 g, 40.9 mmol, 1.0 eq), K₂CO3 (15.3 g, 110.5 mol, 2.7 eq) and 2-bromopropane (6.5 g, 53.2 mmol, 1.3 eq). The reaction was heated to 70° C. overnight. TLC (PE:EA=10:1, R_{f}=0.5) showed the reaction was completed. The reaction was allowed to cool to room temperature. The reaction mixture was poured onto 500 mL crush ice, the mixture was stirred for 10 min, extracted with EA (150 mL*3). The combined organic layers were washed with 10% LiCl aqueous solution (150 mL*2) and dried over Na₂SO₄, filtered and concentrated in vacuum. The filtrate was concentrated in vacuum and purified by column chromatography on silica gel eluted with PE:EA=50:1 to yield the title product (6 g, HPLC: 94.8%) as a light yellow oil. (Yield: 89.5%). Prepare B57: B57 was prepared from 3-isopropoxy-benzaldehyde according the procedure 2, 0.085 g of off-white solid was obtained (Yield: 5.6%). MS-ES: [M+H]⁺:468.7. ¹H NMR (400 MHz, CDCl₃) δ 7.61 (dd, 1H), 7.44 (d, 2H), 7.23-7.14 (m, 3H), 6.99-6.92 (m, 1H), 6.85 (td, 1H), 6.77-6.69 (m, 3H), 4.56 (m, 1H), 4.38 (m, 1H), 2.77-2.68 (m, 2H), 2.53-2.42 (m, 1H), 2.40 (s, 3H), 2.02 (dt, 1H), 1.94-1.82 (m, 1H), 1.75-1.60 (m, 2H), 1.46 (dt, 1H), 1.34 (dd, 6H).

7-Fluoro-2-(4-methoxyphenethyl)-1-tosyl-1,2,3,4-tetrahydroquinoline (B59) 0.080 g of light yellow solid (Yield: 3.0%). MS-ES: [M+H]⁺:440.6. ¹H NMR (400 MHz, CDCl3) δ 7.62 (dd, 1H), 7.44 (d, 2H), 7.19 (d, 2H), 7.11 (d, 2H), 6.96-6.92 (m, 1H), 6.88-6.80 (m, 3H), 4.41-4.33 (m, 1H), 3.80 (s, 3H), 2.71-2.68 (m, 2H), 2.50-2.43 (m, 1H), 2.40 (s, 3H), 2.05-1.96 (m, 1H), 1.89-1.81 (m, 1H), 1.69-1.60 (m, 2H), 1.51-1.41 (m, 1H).

7-Fluoro-2-(2-fluoro-4-methylphenethyl)-1-tosyl-1,2,3,4-tetrahydroquinoline (B60) 0.07 g of white solid (Yield: 8.8%). MS-ES: [M+H]⁺=442.7. ¹H NMR (400 MHz, CDCl₃) δ 7.60 (dd, 1H), 7.44 (d, 2H), 7.20 (d, 2H), 7.09 (t, 1H), 6.97-6.92 (m, 1H), 6.89-6.79 (m, 3H), 4.42-4.34 (m, 1H), 2.77-2.69 (m, 2H), 2.52-2.43 (m, 1H), 2.40 (s, 3H), 2.32 (s, 3H), 2.01 (dt, 1H), 1.87-1.77 (m, 1H), 1.73-1.61 (m, 2H), 1.52-1.44 (m, 1H).

7-Fluoro-2-(2-fluoro-4-methoxyphenethyl)-1-tosyl-1,2,3,4-tetrahydroquinoline (B61) 0.60 g of yellow solid (Yield: 25.0%). MS-ES: =458.7. ¹H NMR (400 MHz, CDCl₃) δ 7.61 (dd, 1H), 7.44 (d, 2H), 7.20 (d, 2H), 7.12 (t, 1H), 6.96 (t, 1H), 6.85 (t, 1H), 6.63 (d, 11), 6.58 (d, 111), 4.46-4.32 (m, 1H), 3.79 (s, 3H), 2.71 (t, 211), 2.55-2.35 (m, 4H), 2.01 (dt, 1H), 1.90-1.78 (m, 1H), 1.75-1.62 (m, 2H), 1.49 (dt, 1H).

2-(4-Chlorophenethyl)-7-fluoro-1-tosyl-1,2,3,4-tetrahydroquinoline (B62) 0.26 g of off-white solid (Yield: 22.6%). MS-ES: [M+H]⁺=444.6. ¹H NMR (400 MHz, CDCl₃) δ 7.61 (dd, 11-), 7.43 (d, 211), 7.25 (d, 2H), 7.20 (d, 2H), 7.12 (d, 211), 6.95 (t, 1H), 6.86 (td, 1H), 4.40-4.32 (m, 1H), 2.74 (t, 2H), 2.51-2.42 (m, 1H), 2.40 (s, 3H), 1.99 (dt, 1H), 1.91-1.80 (m, 1H), 1.70-1.61 (m, 2H), 1.44 (dt, 1H).

7-Fluoro-1-tosyl-2-[4-(trifluoromethyl)phenethyl]-1,2,3,4-tetrahydroquinoline (B63) 0.09 g of white solid (Yield: 7.3%). MS-ES: [M+H]⁺=478.7. ¹H NMR (400 MHz,) δ 7.61 (dd, 1H), 7.54 (d, 2H), 7.43 (d, 2H), 7.31 (d, 2H), 7.20 (d, 2H), 6.96 (t, 1H), 6.86 (td, 1H), 4.42-4.34 (m, 1H), 2.83 (t, 2H), 2.52-2.42 (m, 1H), 2.40 (s, 3H), 2.03-1.95 (m, 1H), 1.95-1.83 (m, 1H), 1.76-1.62 (m, 2H), 1.49-1.41 (m, 1H).

7-Fluoro-1-tosyl-2-[3-(trifluoromethyl)phenethyl]-1,2,3,4-tetrahydroquinoline (B64) 0.14 g of light yellow oil (Yield: 4.7%). MS-ES: [M+H]⁺=478.1. ¹H NMR (400 MHz, DMSO) δ 7.57-7.47 (m, 4H), 7.41-7.32 (m, 5H), 7.15-7.08 (m, 1H), 7.00 (td, 1H), 4.34-4.26 (m, 1H), 2.75 (t, 2H), 2.56-2.45 (m, 1H), 2.35 (s, 3H), 1.92 (dt, 1H), 1.75-1.72 (m, 2H), 1.63-1.43 (m, 2H).

7-Fluoro-2-[2-fluoro-4-(trifluoromethyl)phenethyl]-1-tosyl-1,2,3,4-tetrahydroquinoline (B65) 0.065 g of light yellow oil (Yield: 3.9%). MS-ES: [M+H]⁺=496.7. ¹H NMR (400 MHz, DMSO) δ 7.61-7.46 (m, 3H), 7.45-7.30 (m, 5H), 7.15-7.08 (m, 1H), 7.01 (td, 1H), 4.34 (dq, 1H), 2.85-2.66 (m, 2H), 2.36 (s, 3H), 2.04-1.87 (m, 1H), 1.78-1.44 (m, 4H).

7-Fluoro-2-(3-hydroxyphenethyl)-1-tosyl-1,2,3,4-tetrahydroquinoline (B66) To a 10 mL three-necked flask was added 1 mL DCM and B45 (100 mg, 0.228 mmol, 1.0 eq). The mixture was cooled to −50° C. with nitrogen. Then BBr₃ (285 mg, 1.139 mmol, 5.0 eq) was added to the mixture quickly. The reaction was stirred at RT for 1 h. Then 1 mL ethanol was added dropwise to the mixture at 0° C. The reaction mixture was concentrated under vacuum and purified twice by column chromatography on silica gel eluted with PE:EA=5:1 to afford the title compound B66 30 mg as a white solid. Yield: 31.2%. MS-ES: [M−H]⁻=424.0. ¹H NMR (400 MHz, CDCl₃) δ 7.61 (dd, 1H), 7.43 (d, 2H), 7.20 (d, 2H), 7.17-7.12 (m, 1H), 6.98-6.93 (m, 1H), 6.85 (td, 1H), 6.75 (d, 1H), 6.70-6.66 (m, 2H), 4.42-4.33 (m, 1H), 2.74-2.69 (m, 2H), 2.52-2.44 (m, 1H), 2.40 (s, 3H), 2.05-1.97 (m, 1H), 1.93-1.82 (m, 1H), 1.73-1.61 (m, 2H), 1.50-1.41 (m, 1H).

2-(4-Chloro-2-fluorophenethyl)-1-tosyl-1,2,3,4-tetrahydro-1,5-naphthyridine (B68) Prepare 2-methyl-[1,5]naphthyridine: Step1: To a 250 mL autoclave was added 100 mL MeOH, 2-methyl-5-nitropyridine (15 g, 0.109 mol, 1.0 eq) and 10% Pd/C (0.5 g, 3.3 wt %). The reaction mixture was stirred at RT under 4 atm of hydrogen for 18 h. TLC (PE:EA=3:1) showed the reaction was completed. The reaction mixture was filtered. The filtrate was concentrated in vacuum to afford the title compound 6-methyl-pyridin-3-ylamine (11.5 g, HPLC: 97%) as a dark red solid. Yield: 98.3%. Step2: To a 250 mL round-bottom flask was added concentrated sulfuric acid (66.9 g, 0.68 mol, 11 eq), boric acid (3.84 g, 0.062 mol, 1.0 eq), sodium m-nitrobenzenesulfonate (27.9 g, 0.12 mol, 2.0 eq) and iron sulfate heptahydrate (2.24 g, 8.06 mmol, 0.13 eq). The reaction mixture was stirred at room temperature. Glycerol (28.5 g, 0.31 mol, 5.0 eq) was added to the mixture followed by 6-methyl-pyridin-3-ylamine (6.7 g, 0.062 mol, 1.0 eq) and 30 mL water. The mixture was heated at 135° C. for 18 h. The reaction mixture was cooled to room temperature, basified with 4 N aqueous sodium hydroxide to pH=10 and extracted with EA (50 mL*3). The combined organic layers were dried over Na2SO4, filtered and concentrated in vacuum. The residue was purified by column chromatography on silica gel eluted with PE:EA=5: 1-3:1 to afford the title compound 2-methyl-[1,5]naphthyridine (4.0 g, HPLC: 97%) as an orange oil. Yield: 44.9%.

Prepare B68: B68 was prepared from 2-methyl-[1,5]naphthyridine according the procedure 2, 0.059 g of white solid was obtained (Yield: 1.3%). MS-ES: [M+H]⁺=445.6. ¹H NMR (400 MHz, CDCl₃) δ 8.36 (dd, 1H), 8.19 (d, 1H), 7.37 (d, 2H), 7.25-7.17 (m, 3H), 7.14 (t, 1H), 7.02 (ddd, 2H), 4.35-4.27 (m, 1H), 2.81-2.63 (m, 3H), 2.43-2.33 (m, 4H), 1.83-1.61 (m, 2H), 1.61-1.52 (m, 2H).

2-(4-Chloro-2-fluorophenethyl)-1-tosyl-1,2,3,4-tetrahydroquinoline (B69) 0.61 g of white solid (Yield: 19.0%). MS-ES: [M+H]*=444.6. ¹H NMR (400 MHz, CDCl₃) δ 7.77 (d, 1H), 7.35 (d, 2H), 7.23 (d, 1H), 7.20-7.08 (m, 4H), 7.07-6.92 (m, 3H), 4.32 (m, 1H), 2.86-2.63 (m, 2H), 2.42 (m, 1H), 2.37 (s, 3H), 2.01-1.61 (m, 4H), 1.40 (m, 1H).

2-(4-Chloro-2-fluorophenethyl)-7-methyl-1-tosyl-1,2,3,4-tetrahydroquinoline (B70) and 2-(4-Chloro-2-fluorophenethyl)-5-methyl-1-tosyl-1,2,3,4-tetrahydroquinoline (B70′)

Prepare the mixture of 2,7-Dimethyl-quinoline and 2,5-Dimethyl-quinoline (ratio 1:1): m-tolylamine (92 mmoL) was added to a solution of 37% HCl (200 mL) at 0° C. Paraldehyde (11 mL, 0.8 mol, 9 eq) was then introduced and the mixture was left to react at room temperature for 1 h, and then heated to refluxed for 3 h. After cooling to 0° C., NaOH was slowly added to the solution and extracted with DCM (50 mL*2). The organic layer was dried and concentrated in vacuum and purified by column chromatography on silica gel eluted to afford the mixture 2,7-Dimethyl-quinoline and 2,5-Dimethyl-quinoline (4 g, yield: 28%).

Prepare the mixture of B70 and B70′: The mixture of B70 and B70′ (ratio 1:1) was prepared from the mixture of 2,7-Dimethyl-quinoline and 2,5-Dimethyl-quinoline according the procedure 2, 0.077 g of off-white solid was obtained (Yield: 2.8%). MS-ESI: [M+H]⁺=458.1.

2-(4-Chloro-2-fluorophenethyl)-6-fluoro-1-tosyl-1,2,3,4-tetrahydroquinoline (B71) 0.15 g of white solid (Yield: 12.9%). MS-ES: [M+H]⁺=462.1. ¹H NMR (400 MHz, CDCl₃) δ 7.75 (dd, 1H), 7.38-7.35 (m, 2H), 7.10-6.93 (m, 3H), 6.72 (dd, 1H), 4.35-4.29 (m, 1H), 2.85-2.65 (m, 2H), 2.48-2.32 (m, 4H), 1.92-1.63 (m, 4H), 1.43-1.36 (m, 1H).

2-(4-Chloro-2-fluorophenethyl)-6-methoxy-1-tosyl-1,2,3,4-tetrahydroquinoline (B72) 0.10 g of white solid (Yield: 8.1%). MS-ES: [M+H]⁺=474.8. ¹H NMR (400 MHz, CDCl₃) δ7.66 (d, 1H), 7.34 (d, 2H), 7.16 (t, 3H), 7.06-6.98 (m, 2H), 6.80 (dd, 1H), 6.50 (d, 1H), 4.31-4.23 (m, 1H), 3.79 (s, 3H), 2.82-2.64 (m, 2H), 2.37 (s, 3H), 2.36-2.28 (m, 1H), 1.83-1.63 (m, 4H), 1.38-1.29 (m, 1H).

4-[2-(7-Fluoro-1-tosyl-1,2,3,4-tetrahydro-2-quinolyl)ethyl]benzonitrile (B75) 0.05 g of light yellow solid (Yield: 2.2%). MS-ES: [M+H]⁺=434.9. ¹H NMR (400 MHz, CDCl₃) δ 7.62-7.53 (m, 3H), 7.42-7.37 (m, 2H), 7.31-7.27 (m, 2H), 7.17 (d, 2H), 6.97-6.91 (m, 1H), 6.84 (td, 1H), 4.40-4.31 (m, 1H), 2.87-2.78 (m, 2H), 2.49-2.39 (m, 1H), 2.38 (s, 3H), 1.97 (dt, 1H), 1.91-1.79 (m, 1H), 1.74-1.58 (m, 2H), 1.45-1.38 (m, 1H).

4-[[2-(4-Chloro-2-fluorophenethyl)-7-fluoro-3,4-dihydroquinolin-1(2H)-yl]sulfonyl]benzonitrile (B76)

0.11 g of white solid (Yield: 24.0%). MS-ES: [M+H]⁺=474.8. ¹H NMR (400 MHz, CDCl₃) δ 7.74-7.68 (m, 2H), 7.68-7.62 (m, 2H), 7.60 (dd, 1H), 7.17 (t, 1H), 7.11-6.97 (m, 3H), 6.92 (td, 1H), 4.42-4.33 (m, 1H), 2.83-2.67 (m, 2H), 2.56-2.44 (m, 1H), 1.97-1.62 (m, 4H), 1.54-1.47 (m, 1H).

Procedure 3 (B5, B13, B14, B15, B16, B18′, B27)

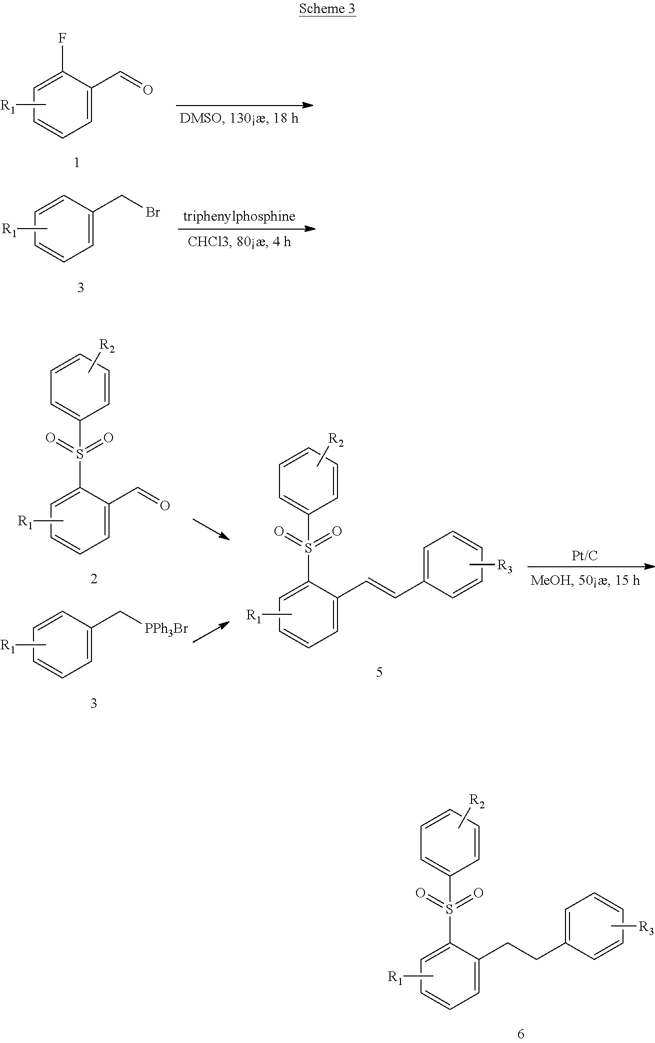

Step 1: To a stirred solution of substituent fluorobenzaldehyde (1.0 equiv.) in dimethyl sulfoxide at room temperature was added substituent sodium benzenesulfonate (1.2 equiv.). The reaction mixture was stirred at 130° C. for 18 hours. After the mixture was cooled down to room temperature, water and ethyl acetate was added to the reaction mixture. The mixture was extracted one more time with ethyl acetate, and the combined organic layers were dried over anhydrous sodium sulfate. The filtrate was concentrated in vacuum and used for next step without purification.

Step 2: Benzyl bromide (1.0 equiv.) and triphenylphosphine (1.1 equiv.) in chloroform was heated under reflux for 4 hours. The solution was cooled to room temperature and the phosphonium was crunched with ether (5V). The crude product was diluted with dichloromethane (1V) and crunched with ether (5V). The precipitated solid was filtered and washed with ether (1V) to afford compound 4 as a white solid (quant. yield).

Step 3: To compound 4 (1.0 equiv.) suspended in tetrahydrofuran was added 60% sodium hydride (1.4 equiv.) over 10 mins at 0° C. After stirring for 1 hour, compound 2 (1.67 equiv.) was added and the mixture was warmed to room temperature for 18 hours. A saturated ammonium chloride aqueous solution was carefully added. The organic layer was separated, and the aqueous layer was extracted with ethyl acetate twice. The combined organic layers were washed with brine, dried over anhydrous sodium sulfate. The filtrate was concentrated in vacuum and purified by silica gel column chromatography to afford compound 5 as a white to yellow solid.

Step 4: A mixture of compound 5 and Pt/C in methanol was stirred at 50° C. under atmospheric pressure of hydrogen for 15 hours. The catalyst and solvent were removed and then the residue was purified by silica gel column chromatography to give compound 6 as viscous oil or a white solid.

1-Phenethyl-2-(phenylsulfonyl)benzene (B5): 5 g of colorless oil (Yield: 64.1%). MS-EI: [M]⁺=322.1. ¹H NMR (300 MHz, CDCl₃): 8.25 (t, 1H), 7.88 (t, 2H), 7.61-7.41 (m, 5H), 7.30-7.17 (m, 6H), 3.14 (q, 2H), 2.78 (q, 2H).

4-Chloro-2-fluoro-1-(2-tosylphenethyl)benzene (B13): 3.2 g of off-white solid (Yield: 64.5%). MS-ESI: [M+1]⁺=388.9. ¹H NMR (300 MHz, CDCl₃): 8.19 (t, 1H), 7.74 (t, 2H), 7.52-7.42 (m, 2H), 7.30-7.22 (m, 3H), 7.10-7.04 (m, 3H), 3.10 (q, 2H), 2.84 (q, 2H), 2.42 (s, 3H).

4-Chloro-1-[2-[(4-chlorophenyl)sulfonyl]phenethyl]-2-fluorobenzene (B14): 1.1 g of yellow solid (Yield: 21.3%). MS-ESI: [M+1]⁺=425.9. ¹H NMR (300 MHz, CDCl₃): 8.21 (t, 1H), 7.79 (t, 2H), 7.58-7.42 (m, 4H), 7.28-7.25 (m, 1H), 7.14-7.05 (m, 3H), 3.08 (q, 2H), 2.88 (q, 2H).

2-(4-Chloro-2-fluorophenethyl)-1-tosylnaphthalene (B15): 0.35 g of off-white solid (Yield: 4.4%). MS-ESI: [M+1]⁺=438.9. ¹H NMR (300 MHz, CDCl₃): 8.89 (t, 1H), 7.98 (t, 2H), 7.85-7.75 (m, 3H), 7.56-7.47 (m, 2H), 7.40-7.20 (m, 5H), 7.11-7.07 (m, 2H), 3.64 (q, 2H), 3.17 (q, 2H), 2.37 (s, 3H).

2-(4-Chloro-2-fluorophenethyl)-1-[(4-chlorophenyl)sulfonyl]naphthalene (B16): 0.5 g of off-white solid (Yield: 3.7%). MS-ES: [M+1]⁺=458.8. ¹H NMR (300 MHz, CDCl₃): 8.81 (t, 1H), 8.01 (t, 2H), 7.87-7.78 (m, 3H), 7.58-7.49 (m, 2H), 7.46-7.38 (m, 3H), 7.32-7.26 (m, 1H), 7.12-7.07 (m, 2H), 3.62 (q, 2H), 3.17 (t, 2H).

3-(4-Chloro-2-fluorophenethyl)-4-tosylpyridine (B27): 0.5 g of yellow oil (Yield: 0.72%). MS-ESI: [M+1]⁺=390.4. ¹H NMR (300 MHz, CDCl₃): 8.70 (t, 1H), 8.48 (s, 1H), 7.87-7.78 (m, 3H), 7.37-7.21 (m, 2H), 7.14-7.06 (m, 3H), 3.16 (q, 2H), 2.89 (t, 2H), 2.45 (s, 3H).

Procedure 4 (B4-1, 2, 3, 4)

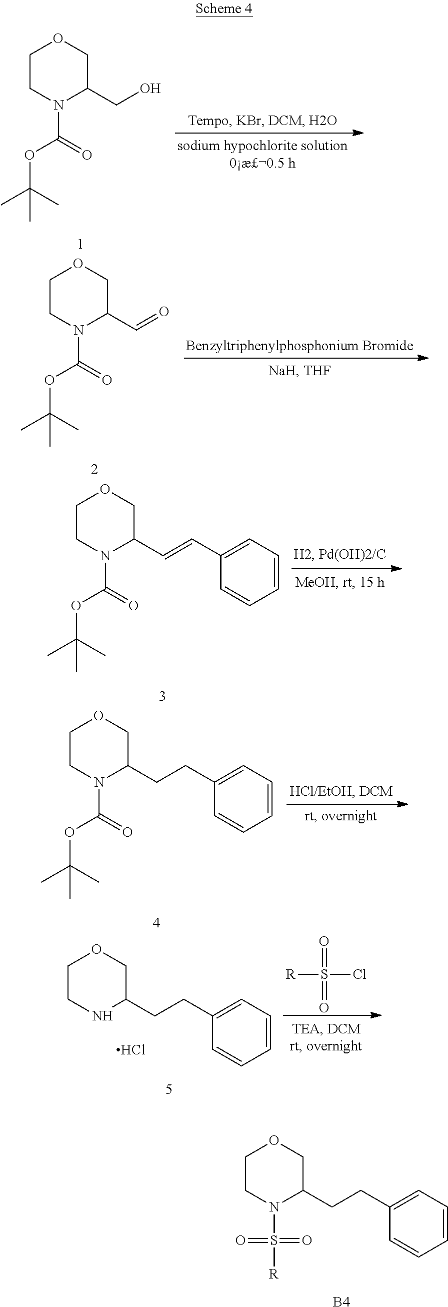

Step 1. To a solution of compound 1 4.25 g (1 eq) and TEMPO 0.06 g (0.02 eq) in dichloromethane (80 ml), was add KBr 1.17 g (0.5 eq), H₂O 27 ml and NaHCO₃8.34 g (4 eq). The mixture was cooled to 10° C. and the 5% sodium hypochlorite solution 53 g (2 eq) was added drop wise. After stirring 0.5 h at 10° C., the mixture was extracted three times with dichloromethane. The combined organic layers was washed with 50 ml of 10% Na₂SO₃ aq., sodium bicarbonate solution and brine, dried over Na₂SO₄ and concentrated to give crude compound 2.

Step 2. To a solution of benzyltriphenylphosphonium bromide 14.3 g (1.7 eq) in tetrahydrofuran (65 ml), was added NaH 1.56 g (60%, 2 eq) at room temperature. The mixture was stirred for 1 h and cooled to 10° C. and crude compound 2 was added.

After stirring overnight at room temperature, saturated aqueous ammonium chloride (100 ml) was added. The organic phases were dried over Na₂SO₄ and concentrated, and the residue was purified by silica column chromatography to give compound 3 as colorless oil (2 g, yield of 2 steps 36%).

Step 3. The mixture of compound 3 (1.5 g) was dissolved in methanol (100 ml). 10% Pd (OH)₂/C (0.3 g) was added. Hydrogenation was carried out under the pressure of 30 bars at the room temperature for 15 h. The catalyst was filtered and washed three times with methanol (3×20 mL). The filtrates were combined and concentrated under vacuum, compound 4 (1.5 g, 100 percent) was obtained as colorless oil.

Step 4. The mixture of compound 4 (1.5 g, 1 eq) was dissolved in dichloromethane (30 ml). 30 percent HCl/EtOH (1.88 g, 3 eq) was added, then stirring overnight at room temperature, concentrated under vacuum, compound 5 (1.52 g, yield 90%) was obtained as oil. MS-ESI: [M−HCl+H]⁺=192.

Step 5. The mixture of compound 5 (1 eq) and triethylamine (3 eq) was dissolved in dichloromethane. The corresponding sulfonyl chloride (1 eq) was added. After stirring overnight at room temperature, the solution were concentrated under vacuum, and the residue was purified by silica column chromatography to give compound B4.

3-Phenethyl-4-(phenylsulfonyl)morpholine (B4-1): 0.3 g of off-white powder (Yield: 18%). MS-ESI: [M+1]=332.5. ¹H NMR (300 MHz, CDCl₃): δ 7.82 (d, 2H), 7.63-7.49 (m, 3H), 7.63-7.49 (m, 3H), 7.32-7.28 (m, 2H), 7.23-7.11 (m, 3H), 3.90-3.56 (m, 4H), 3.50-3.26 (m, 3H), 2.62 (t, 2H), 2.105-1.824 (m, 2H).

4-[(4-Fluorophenyl)sulfonyl]-3-phenethylmorpholine (B4-2): 0.39 g of off-white powder (Yield: 21%). MS-ESI: [M+1]=350.5. ¹H NMR (300 MHz, CDCl₃): δ 7.86-7.77 (m, 2H), 7.35-7.27 (m, 2H), 7.26-7.12 (m, 5H), 3.87-3.71 (m, 3H), 3.60 (d, 1H), 3.50-3.24 (m, 3H), 2.72-2.54 (m, 2H), 2.14-2.01 (m, 1H), 1.94-1.82 (m, 1H).

4-[(4-Methoxyphenyl)sulfonyl]-3-phenethylmorpholine (B4-3): 0.41 g of off-white powder (Yield: 21%). MS-ESI: [M+1]=362.5. ¹H NMR (300 MHz, CDCl₃): δ 7.75 (d, 2H), 7.34-7.26 (m, 2H), 7.25-7.13 (m, 3H), 6.98 (d, 2H), 3.89 (s, 3H), 3.86-3.67 (m, 3H), 3.59 (d, 1H), 3.50-3.24 (m, 3H), 2.63 (t, 2H), 2.11-1.85 (m, 2H).

3-Phenethyl-4-tosylmorpholine (B4-4): 0.51 g of off-white powder (Yield: 27%). MS-ESI: [M+1]=346.5. ¹H NMR (300 MHz, CDCl₃): δ 7.70 (d, 2H), 7.36-7.26 (m, 4H), 7.25-7.12 (m, 3H), 3.88-3.55 (m, 4H), 3.48-3.24 (m, 3H), 2.63 (t, 2H), 2.45 (s, 3H), 2.09-1.83 (m, 2H).

Procedure for B23

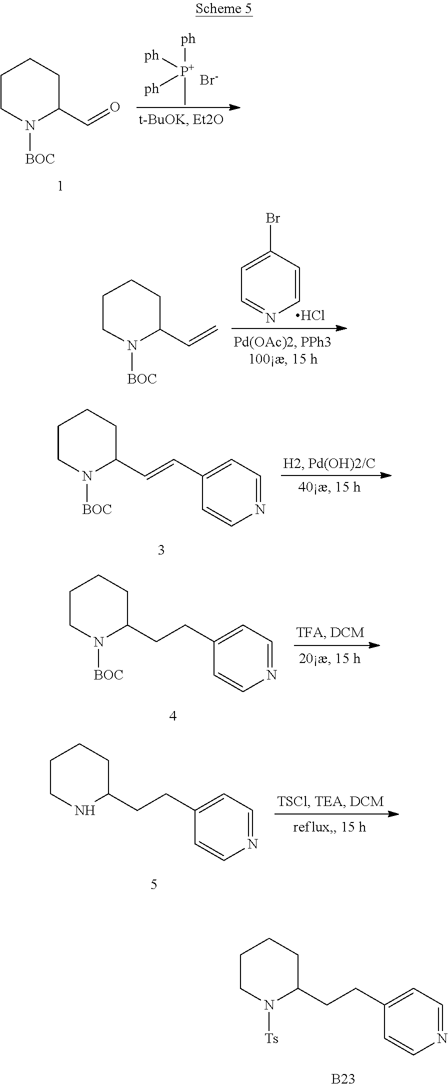

Step 1. The mixture of t-BuOK (3.15 g, 2 eq) and Methyltriphenylphosphonium bromide (10 g, 2 eq) was dissolved in ether (100 ml) and refluxed for 1 h. It was cooled to room temperature, and compound 1 (3 g, 1 eq) was added to the solution. After refluxed 1 h, water (100 ml) was added and the organic phases were dried over Na₂SO₄ and concentrated. The residue was purified by silica column chromatography to give compound 2 as colorless oil (3.1 g).

Step 2. The mixture of compound 2 (3.1 g, 2 eq), triethylamine (7.11 g, 5 eq), 4-Bromopyridine Hydrochloride (5.48 g, 2 eq), Tri(o-tolyl)phosphine (0.86 g, 0.2 eq) and Pd(OAc)₂ (0.32 g, 0.1 eq) were dissolved in DMF (30 ml) was stirred for 4 h at 100° C. under nitrogen atmosphere. Then it was cooled to room temperature, poured to 200 ml water and extracted with dichloromethane. The organic layer was washed with brine, dried over Na₂SO₄ and concentrated. The residue was purified by silica column chromatography to give compound 3 as yellow oil (3.4 g, yield of 2 steps 85%).

Step 3. The mixture of compound 3 (3.4 g) was dissolved in methanol (30 ml). 10% Pd(OH)₂/C (0.5 g) was added. Hydrogenation was carried out under the pressure of 30 bars at 40° C. for 15 h. The catalyst was filtered and washed three times with methanol (3×10 mL). The filtrates were combined and concentrated under vacuum to obtained compound 4 as yellow oil (3.4 g, Yield 100%). MS-ES: [M+1]⁺=291.3.

Step 4. The mixture of compound 4 (3.4 g) was dissolved in dichloromethane (200 ml). Trifluoroacetic acid (15 ml) was added drop wise at 20° C. After stirring for 1 h, the solution were concentrated under vacuum. The residue was dissolve with dichloromethane (100 ml) and methanol (10 ml). After the pH was adjusted to alkaline with sodium bicarbonate, it was concentrated under vacuum to give crude compound 5 (15 g), which was used directly to next step without further purification. MS-ES: [M+1]⁺=191.3.

Step 5. The mixture of crude compound 5 (5 g, 1 eq) and triethylamine (1.2 g, 3 eq) was dissolved in dichloromethane (100 ml). The 4-toluene sulfonyl chloride (0.82 g, 1 eq) was added, and then refluxed overnight, the solution were concentrated under vacuum, and the residue was purified by silica gel column chromatography to give 0.08 g 4-[2-(1-Tosyl-2-piperidinyl)ethyl]pyridine (B23) as yellow oil (Yield: 5%). MS-ES: [M+1]⁺=345.4. ¹H NMR (300 MHz, CDCl₃): δ 8.51 (d, 2H), 7.74 (d, 2H), 7.31 (d, 2H), 7.13 (d, 2H), 4.12 (s, 1H), 3.85 (d, 1H), 3.05 (t, 1H), 2.76-2.60 (m, 2H), 2.45 (s, 3H), 2.13-1.97 (m, 1H), 1.79-1.66 (m, 2H), 1.57-1.39 (m, 5H).

Procedure for B25

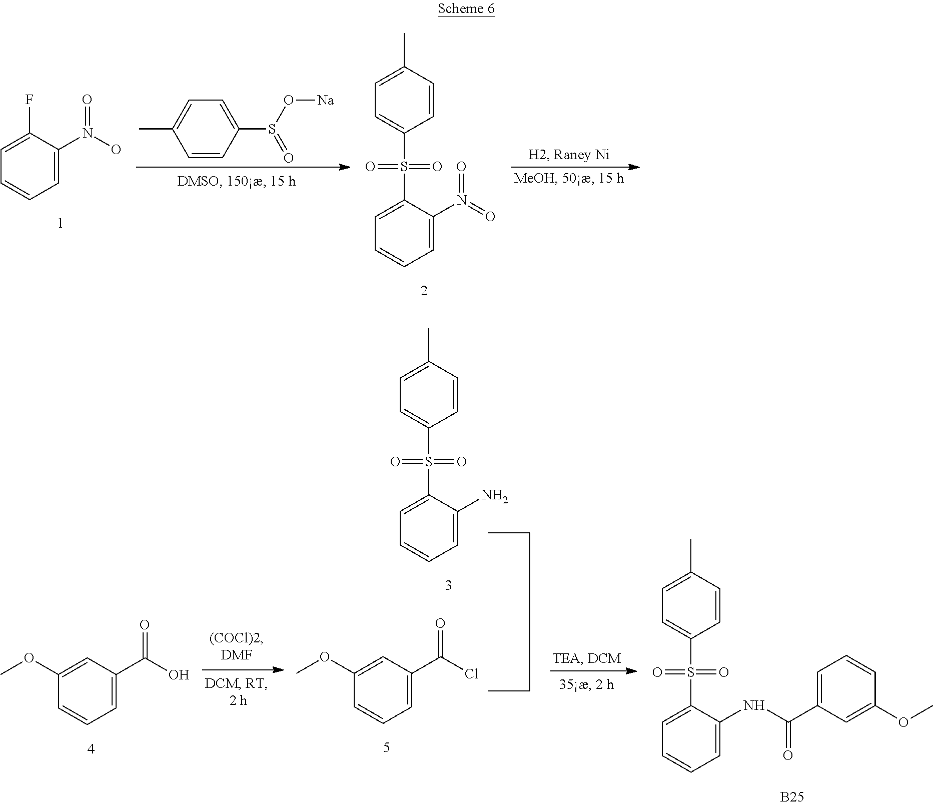

Step 1. The mixture of compound 1 (25 g, 1 eq) and sodium p-tolylsulfinate (47 g, 1.5 eq) was dissolved in DMSO (300 ml). After stirring 15 h at 150° C., the mixture was cooled to room temperature, poured to 200 ml ice water and extracted with ethyl acetate. The organic layer was washed with brine, dried over Na₂SO₄ and concentrated. The residue was recrystallized with petroleum ether and ethyl acetate to give compound 2 (40 g, Yield 81%).

Step 2. The compound 2 (40 g) was dissolved in methanol (400 ml). Raney Ni (10 g) was added. Hydrogenation was carried out under the pressure of 30 bars for 15 h at 50° C. The catalyst was filtered and washed three times with methanol (3×20 mL). The filtrates were combined and concentrated under vacuum to give crude compound 3 (37 g. MS-ES: [M+1]⁺=248.3.

Step 3. The compound 4 (10 g, 1 eq) was dissolved in dichloromethane (30 ml). Oxalyl chloride (9.2 g, 1.1 eq) was added at 10° C., and then 2 drops N, N-dimethylformamide was added. After stirring 15 h at room temperature, the solution was concentrated under vacuum and the residue was used directly in the next step without further purification.

Step 4. The mixture of compound 3 (3.2 g, 1 eq) and triethylamine (3.3 g, 5 eq) was dissolved in dichloromethane (30 ml). compound 5 (1.32 g, 1.2 eq) was added. After reflux for 2 h, the solution were concentrated under vacuum, and the resulting crude product was purified by silica column chromatography to give 3-Methoxy-N-(2-tosylphenyl)benzamide (B25) as yellow solid (0.7 g, yield 12%). MS-ES: [M+1]⁺=382.3. ¹H NMR (300 MHz, CDCl₃): δ 10.66 (s, 1H), 8.63 (d, 1H), 8.06 (d, 1H), 7.73-7.57 (m, 5H), 7.50 (t, 1H), 7.32-7.15 (m, 4H), 3.94 (s, 3H), 2.37 (s, 3H).

Procedure for B26

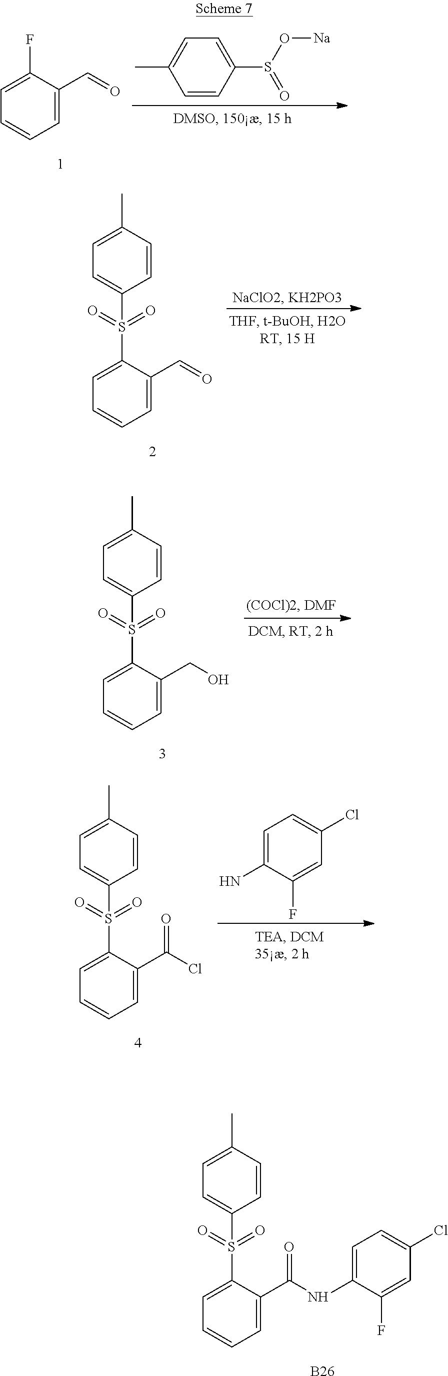

Step1. The mixture of compound 1 (11.6 g, 1 eq) and sodium p-tolylsulfinate (25 g, 1.5 eq) was dissolved in DMSO (150 ml). After stirring 15 h at 150° C., the mixture was cooled to room temperature, poured to 200 ml ice water and extracted with ethyl acetate. The organic layer was washed with brine, dried over Na₂SO₄ and concentrated to give compound 2 (24 g, yield 100%).

Step 2. To a solution of compound 2 (24 g, 1 eq) and KH₂PO₃ (32 g, 2.5 eq) in tetrahydrofuran (400 ml), t-BuOH (200 ml) and H₂O (200 ml), was added NaClO₂(21 g, 2.5 eq) at room temperature. After stirring for 15 h, the mixture was adjusted pH to 9 with 2N sodium hydroxide solution, and extracted with ethyl acetate. The organic layer was adjusted pH to 9 with 2N hydrochloric acid solution. The phases were separated and the organic phase was dried over Na₂SO₄ and concentrated to give compound 2 as a white solid (20 g, yield 80%). MS-ES: [M−1]⁻=275.3.

Step 3. The compound 4 (5 g, 1 eq) was dissolved in dichloromethane (30 ml). Oxalyl chloride (2.5 g, 1.1 eq) was added at 10° C., and then 2 drops N, N-dimethylformamide was added. After stirring 15 h at room temperature, the solution was concentrated under vacuum and the residue was used directly in the next step without further purification.

Step 4. The mixture of compound 3 (6 g, 1 eq) and triethylamine (6 g, 5 eq) was dissolved in dichloromethane (30 ml). 4-Chloro-2-fluoroaniline (1.75 g, 1 eq) was added. After stirring 15 h at 30° C., the solution was added water (100 ml) and extracted with dichloromethane. The organic layer was washed with brine, dried over Na₂SO₄ and concentrated under vacuum. The resulting crude product was recrystallized with petroleum ether and ethyl acetate to give N-(4-Chloro-2-fluorophenyl)-2-tosylbenzamide (B26) as white solid (0.7 g, yield 28%). MS-ES: [M−1]⁻=402.5. ¹H NMR (300 MHz, CDCl₃): δ 8.36 (t, 1H), 8.23-8.16 (m, 1H), 8.00 (s, 1H), 7.82 (d, 2H), 7.73-7.59 (m, 3H), 7.33-7.17 (m, 4H), 2.14 (s, 3H).

Compounds B54 and B77-B91 were synthesized using similar procedures as described above.

### Biological Assays

### Example A1: Endolysosomal Electrophysiology

Endolysosomal electrophysiology was performed in isolated endolysosomes using a modified patch-clamp method. Briefly, cells were treated with 1 μM vacuolin-1 for 2-5 h to increase the size of endosomes and lysosomes. Whole-endolysosome recordings were performed on isolated enlarged LELs. The bath (internal/cytoplasmic) solution contained 140 mM K-gluconate, 4 mM NaCl, 1 mM EGTA, 2 mM Na2-ATP, 2 mM MgCl₂, 0.39 mM CaCl₂), 0.2 mM GTP and 10 mM HEPES (pH adjusted with KOH to 7.2; free [Ca²⁺]i˜100 nM based on the Maxchelator software (http://maxchelator.stanford.edu/)). The pipette (luminal) solution consisted of a ‘Low pH Tyrode's solution with 145 mM NaCl, 5 mM KCl, 2 mM CaCl₂), 1 mM MgCl₂, 10 mM HEPES, 10 mM MES and 10 mM glucose (pH 4.6). All bath solutions were applied via a perfusion system to achieve a complete solution exchange within a few seconds. Data were collected using an Axopatch 2A patch clamp amplifier, Digidata 1440 and pClamp 10.0 software (Axon Instruments). Whole-cell currents were digitized at 10 kHz and filtered at 2 kHz. All experiments were conducted at room temperature (21-23° C.), and all recordings were analyzed with pClamp 10.0 and Origin 8.0 (OriginLab, Northampton, Mass., USA). ML1 channel activity was induced by synthetic agonists ML-SA1 or MS-SA5, and inhibited by ML-S13 or ML-S14.

In this electrophysiology assays, compounds were tested with both basal and agonist-induced ML1 currents. Examples B11, B11a, B11b, B40, B41, B44, B5, B47, B48, B49, B50, B51, B54, B57, B58, B59, B62, B63, B68, B69, B71, B72, B75, B75, and B76 were tested with basal ML1 currents. Many Examples showed significant inhibition of basal ML1 currents. In particular, Examples B11, B11a and B11b inhibited basal ML1 currents with IC50<100 nM. Examples B11, B11a and B11b are shown below. Examples B1-4, B2-1, B-2-2, B2-3, B2-4, 3-4, B5, B6-2, B9-4, B-10, B-10-4, B11, B12, B13, B14, B15, B16, B18, B19, B20, B21, B22, B23, B25, B26, B27, B28, B29, B30, B32, B34, B40, B41, B44, B45, B46, B47, B48, B49, B50, B51, B52, B59, and B66 were tested in ML1 currents activated by ML1 agonists (ML-SA1 or ML-SA5). Many Examples showed significant inhibition of agonist-activated ML1 currents. In particular, B11 inhibited the ML-SA1-activated currents with IC50<1,000 nM. Example B11 is shown below.

### Example A2: Lysosomal Ca²⁺ Imaging

GCaMP3-ML1 expression was induced in Tet-On HEK-GCaMP3-ML1 cells 20-24 h prior to experiments using 0.01p g/mL doxycycline. GCaMP3-ML1 fluorescence was monitored at an excitation wavelength of 470 nm (F₄₇₀) using an EasyRatio Pro system (PTI). Cells were bathed in Tyrode's solution containing 145 mM NaCl, 5 mM KCl, 2 mM CaCl₂), 1 mM MgCl₂, 10 mM Glucose, and 20 mM Hepes (pH 7.4). Lysosomal Ca²⁺ release was measured in a zero Ca²⁺ solution containing 145 mM NaCl, 5 mM KCl, 3 mM MgCl₂, 10 mM glucose, 1 mM EGTA, and 20 mM HEPES (pH 7.4). Ca²⁺ concentration in the nominally free Ca²⁺ solution is estimated to be 1-10 μM. With 1 mM EGTA, the free Ca²⁺ concentration is estimated to be <10 nM based on the Maxchelator software (http://maxchelator.stanford.edu/). Experiments were carried out 0.5 to 6 hrs after plating. ML1-mediated lysosomal Ca²⁺ release was triggered by synthetic agonists ML-SA1, and inhibited by ML-S13 and aforementioned testing compounds.

For example, in this calcium imaging assay, Example B-11b inhibited ML-SA1-induced lysosomal Ca²⁺ release with IC₅₀<1,000 nM as shown below.

### Example A3: Gastric Acid Secretion Assay

Acid secretion was measured in cultured parietal cells upon histamine stimulation. Briefly, after mucosal digestion of isolated glands, supernatants were pelleted by centrifugation at 200×g, washed three times with HEPES-MEM, and re-suspended in Medium A. Approximately 70% of the total gastric cells suspended in Medium A were parietal cells. The cells were plated onto Matrigel-coated 18-mm round coverslips or 35-mm dishes and incubated at 37° C. In cultured parietal cells, upon secretagogue stimulation, apical canalicular membranes are engulfed into the cell to form multiple actin-wrapped vacuoles known as vacuolar apical compartments (VACs), which remain separate from the basolateral membrane and free TVs in the cytosol (Nakada, S. L., Crothers, J. M., Jr., Machen, T. E., and Forte, J. G. (2012). Apical vacuole formation by gastric parietal cells in primary culture: effect of low extracellular Ca2+. American journal of physiology Cell physiology 303, C1301-1311.). Hence total VAC membrane area provides a quantitative measurement of TV exocytosis. In resting WT cells, small (diameter <2 μm) VACs were observed occasionally with a total surface area <10 μm². VACs formed within 10-20 min after bath application of histamine and then fused together to generate one or a few large VACs (up to 8 μm in diameter; total VAC surface area >50 μm²). The apical membrane vacuole surface area during resting and stimulated states was measured for all VACs that were visible in multiple (3-6) Z-cross sections. Data were analyzed in ImageJ (NIH).

In this acid secretion assay, histamine-stimulated VAC formation was abolished by ML-S4 at 10 μM (Zhang X, Cheng X, Yu L, Yang J, Calvo R, Patnaik S, Hu X, Gao Q, Yang M, Lawas M, Delling M, Marugan J, Ferrer M, and Xu H. 2016, PMID: 27357649). In comparison, Examples B11 and B11b significantly inhibited histamine-induced VAC formation at 0.5 μM and abolished it at 2 μM as shown below.

What is claimed:

1. A compound of formula I: 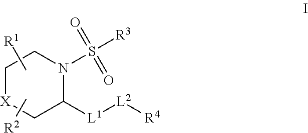
or a salt thereof; or a prodrug, or a salt of a prodrug thereof; or a hydrate, solvate, or polymorph thereof, wherein:
R¹ and R² each are independently H, alkyl, haloalkyl, halogen, oxo, amino, or alkylamino; or R¹ and R² together with the atoms they are bonded form a 5-7 membered aryl, heteroaryl, cycloalkyl, cycloheteroalkyl or partially unsaturated ring optionally substituted with one or more substituents independently selected from the group consisting of halo, cyano, (C₁-C₆)alkyl, (C₁-C₆)haloalkyl, (C₂-C₆)alkenyl, (C₂-C₆)alkynyl, (C₁-C₆)alkoxy, and (C₁-C₆)haloalkoxy;
R³ and R⁴ are each independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, or two substituents together with the atoms they are bonded form a 5-7 membered cycloalkyl, or cycloheteroalkyl optionally substituted with one or more substituents independently selected from the group consisting of halo, and (C₁-C₆)alkyl; wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;
X is CR⁶R⁷, O, SOq wherein q is 0, 1 or 2, or NR⁶; R⁶ and R⁷ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;
L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR; R is an (C₁-C₆)alkyl.

2. A compound according to claim 1, wherein said compound is of formula IA: 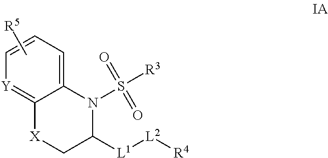
wherein R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, or two substituents together with the atoms they are bonded form a 5-7 membered cycloalkyl, or cycloheteroalkyl optionally substituted with one or more substituents independently selected from the group consisting of halo, and (C₁-C₆)alkyl; wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;
R⁵ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;
X is CR⁶R⁷, O, SOq wherein q is 0, 1 or 2, or NR⁶; R⁶ and R⁷ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;
Y is Nor CR⁸; R⁸ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;
L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR—; R is an (C₁-C₆)alkyl.

3. A compound according to claim 1, wherein said compound is selected from the group consisting of:

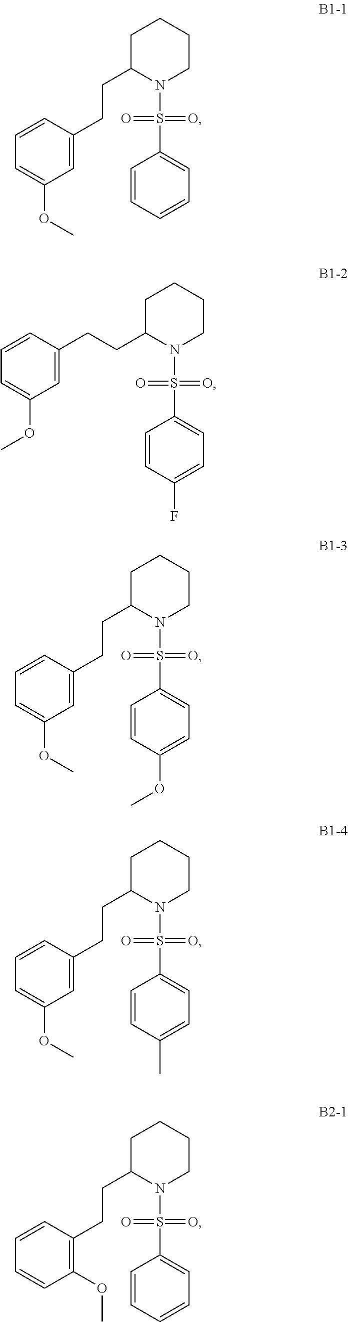

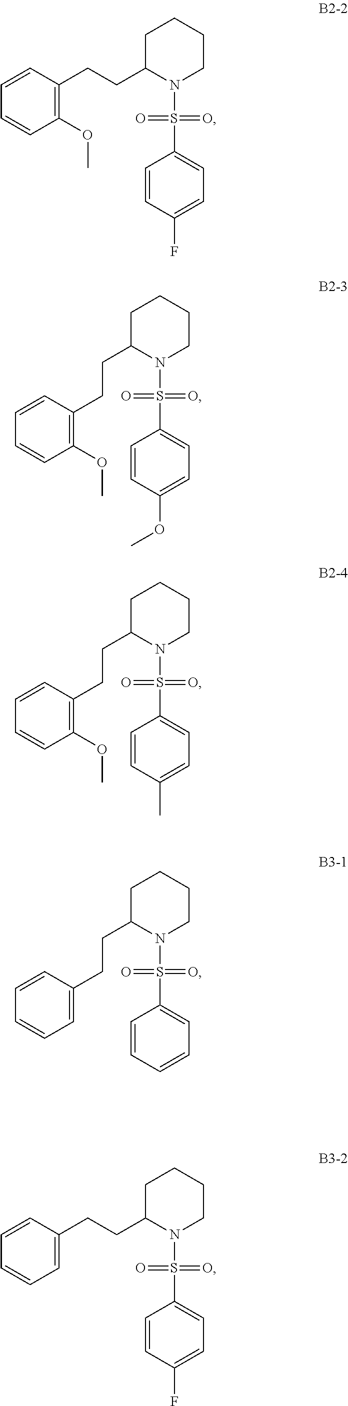

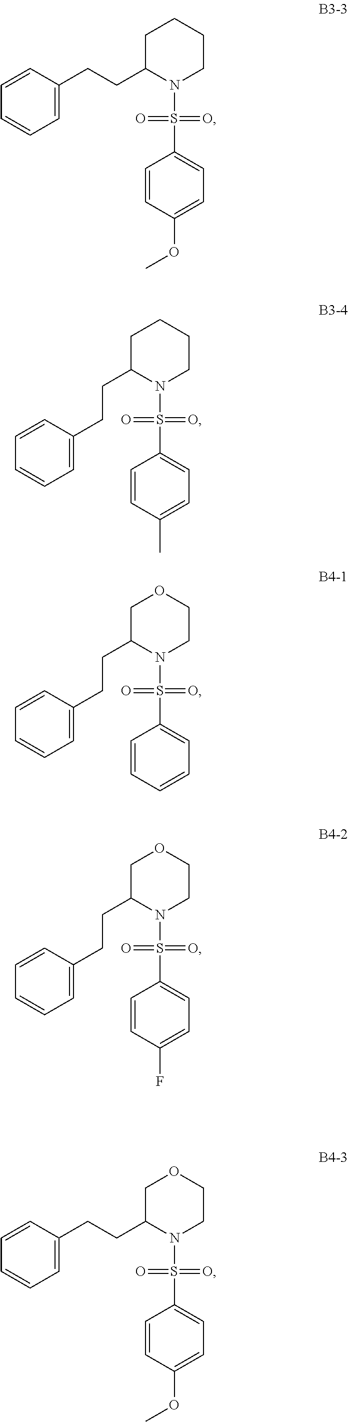

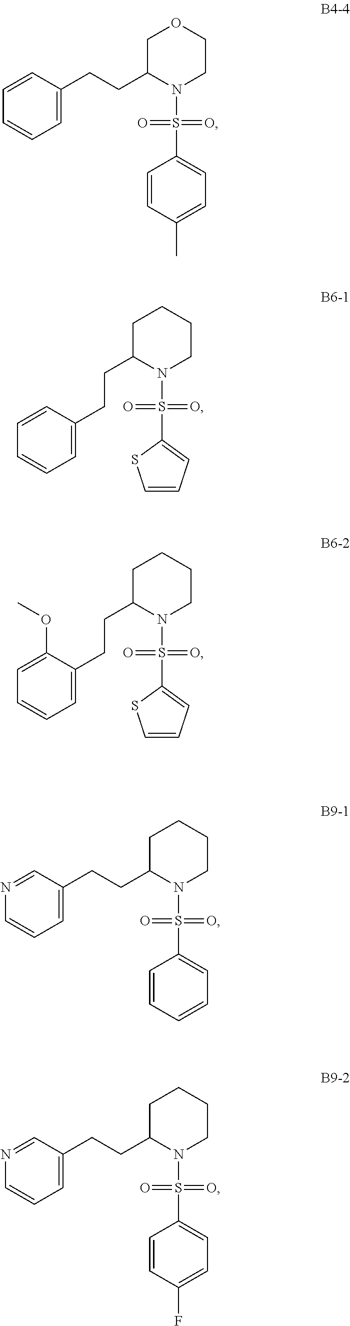 [SMILES: Cc1ccc(S(=O)(=O)N2CCOCC2CCc2ccccc2)cc1 ; O=S(=O)(c1cccs1)N1CCCCC1CCc1ccccc1 ; COc1ccccc1CCC1CCCCN1S(=O)(=O)c1cccs1 ; O=S(=O)(c1ccccc1)N1CCCCC1CCc1cccnc1 ; O=S(=O)(c1ccc(F)cc1)N1CCCCC1CCc1cccnc1]

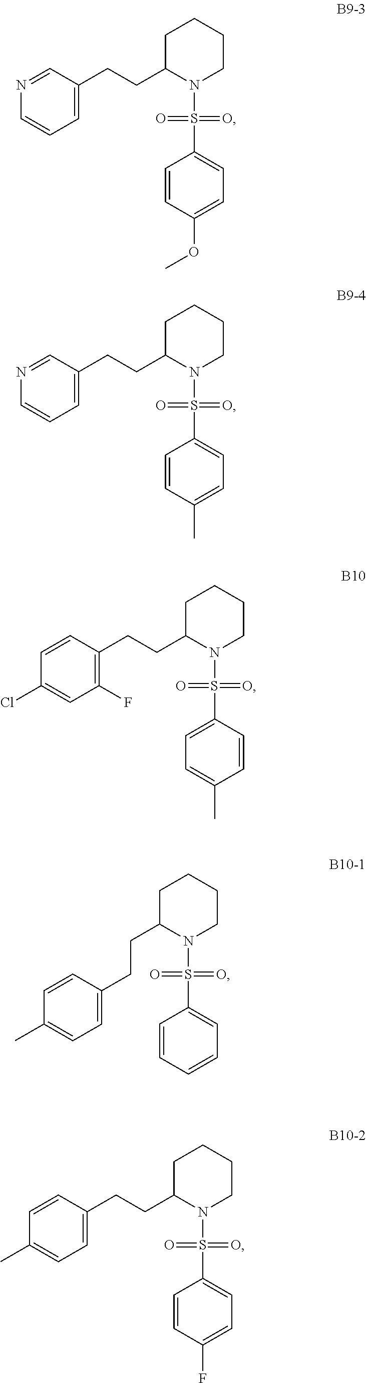

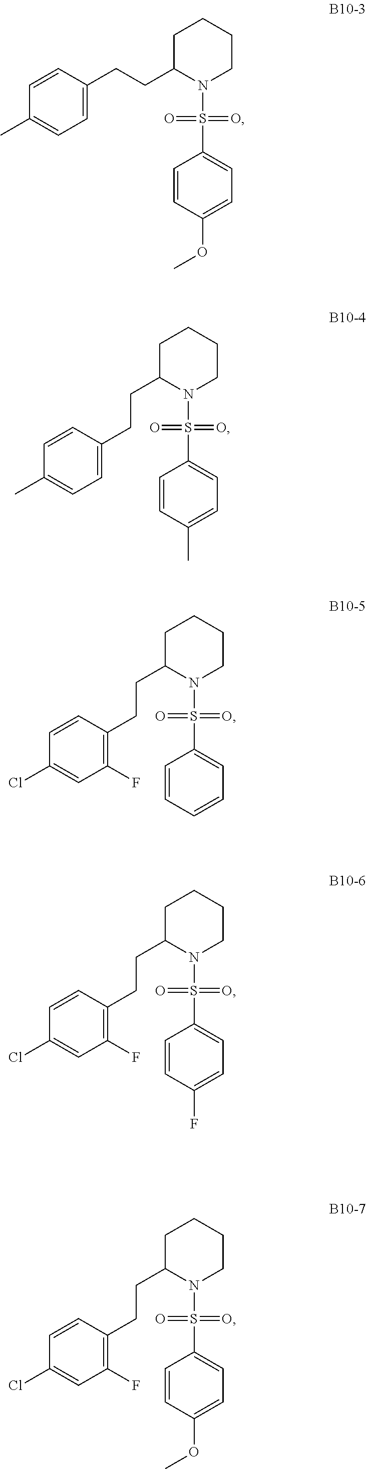 [SMILES: COc1ccc(S(=O)(=O)N2CCCCC2CCc2ccc(C)cc2)cc1 ; Cc1ccc(CCC2CCCCN2S(=O)(=O)c2ccc(C)cc2)cc1 ; O=S(=O)(c1ccccc1)N1CCCCC1CCc1ccc(Cl)cc1F ; O=S(=O)(c1ccc(F)cc1)N1CCCCC1CCc1ccc(Cl)cc1F ; COc1ccc(S(=O)(=O)N2CCCCC2CCc2ccc(Cl)cc2F)cc1]

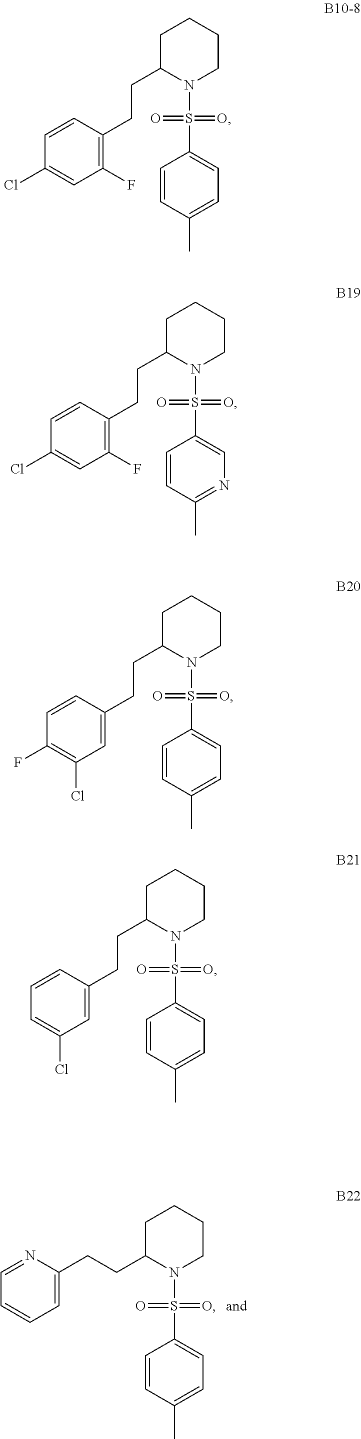

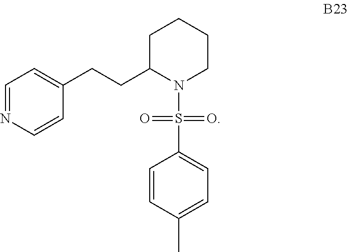 [SMILES: Cc1ccc(S(=O)(=O)N2CCCCC2CCc2ccncc2)cc1]

4. A compound according to claim 2, wherein said compound is selected from the group consisting of:

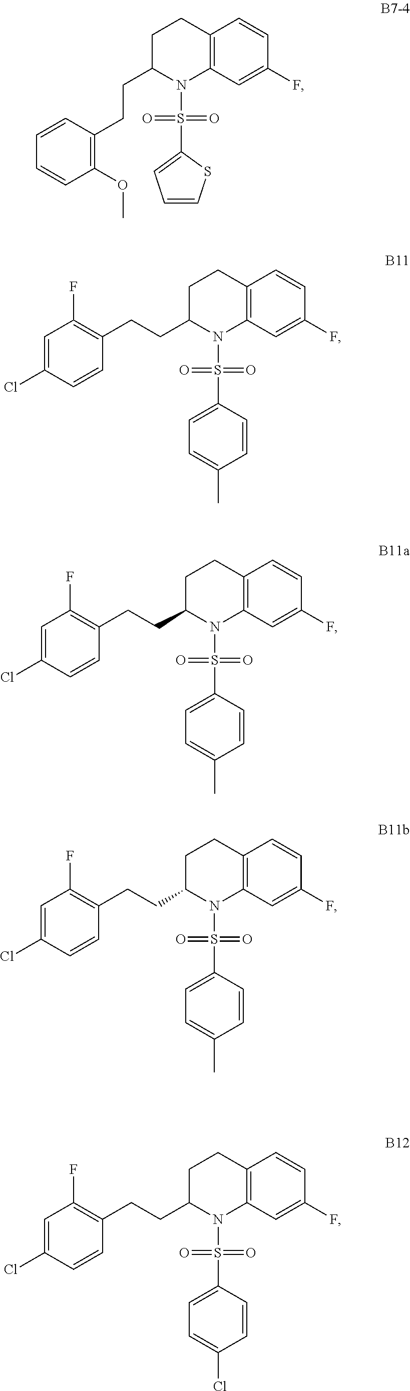

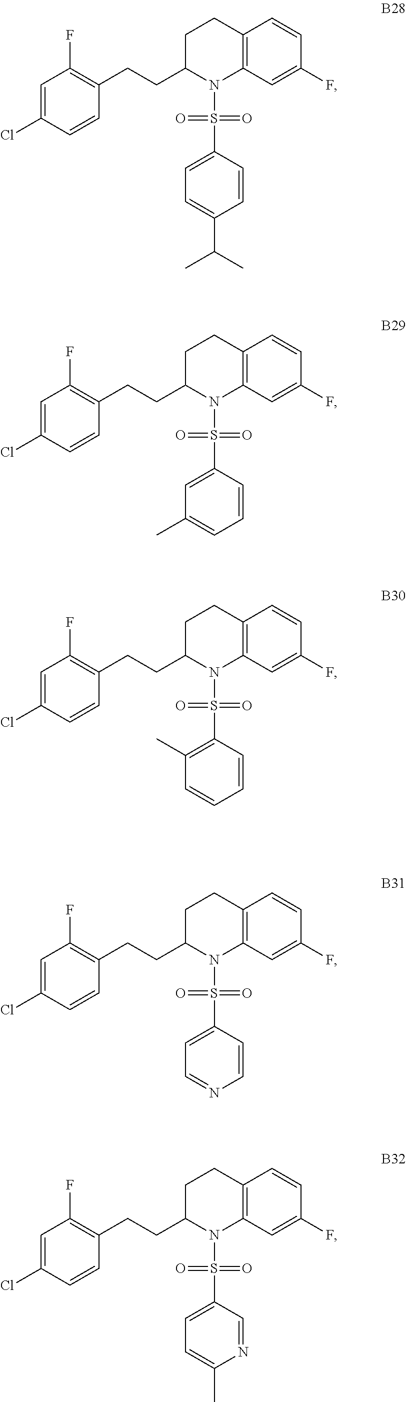 [SMILES: CC(C)c1ccc(S(=O)(=O)N2c3cc(F)ccc3CCC2CCc2ccc(Cl)cc2F)cc1 ; Cc1cccc(S(=O)(=O)N2c3cc(F)ccc3CCC2CCc2ccc(Cl)cc2F)c1 ; Cc1ccccc1S(=O)(=O)N1c2cc(F)ccc2CCC1CCc1ccc(Cl)cc1F ; O=S(=O)(c1ccncc1)N1c2cc(F)ccc2CCC1CCc1ccc(Cl)cc1F ; Cc1ccc(S(=O)(=O)N2c3cc(F)ccc3CCC2CCc2ccc(Cl)cc2F)cn1]

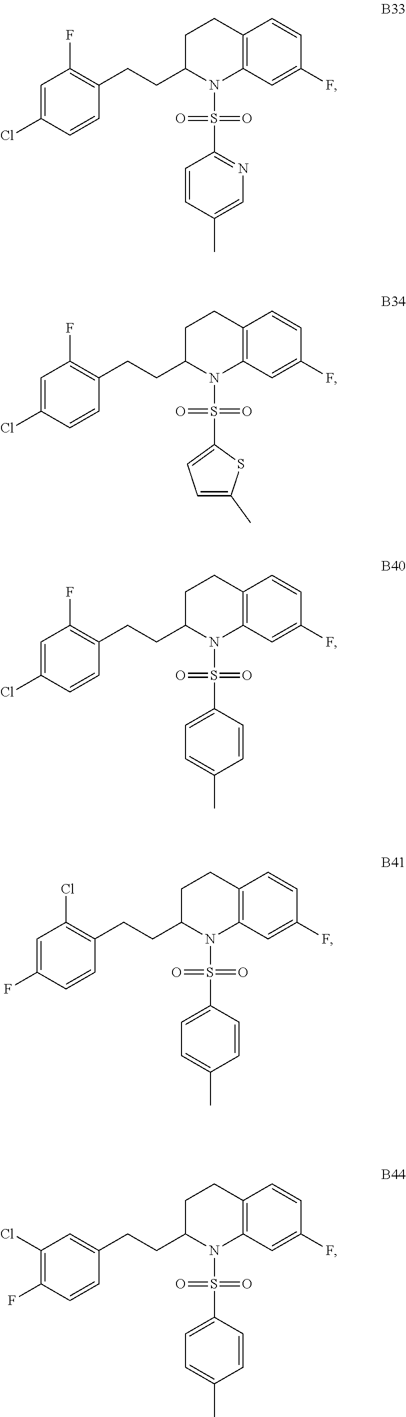 [SMILES: Cc1ccc(S(=O)(=O)N2c3cc(F)ccc3CCC2CCc2ccc(Cl)cc2F)nc1 ; Cc1ccc(S(=O)(=O)N2c3cc(F)ccc3CCC2CCc2ccc(Cl)cc2F)s1 ; Cc1ccc(S(=O)(=O)N2c3cc(F)ccc3CCC2CCc2ccc(Cl)cc2F)cc1 ; Cc1ccc(S(=O)(=O)N2c3cc(F)ccc3CCC2CCc2ccc(F)cc2Cl)cc1 ; Cc1ccc(S(=O)(=O)N2c3cc(F)ccc3CCC2CCc2ccc(F)c(Cl)c2)cc1]

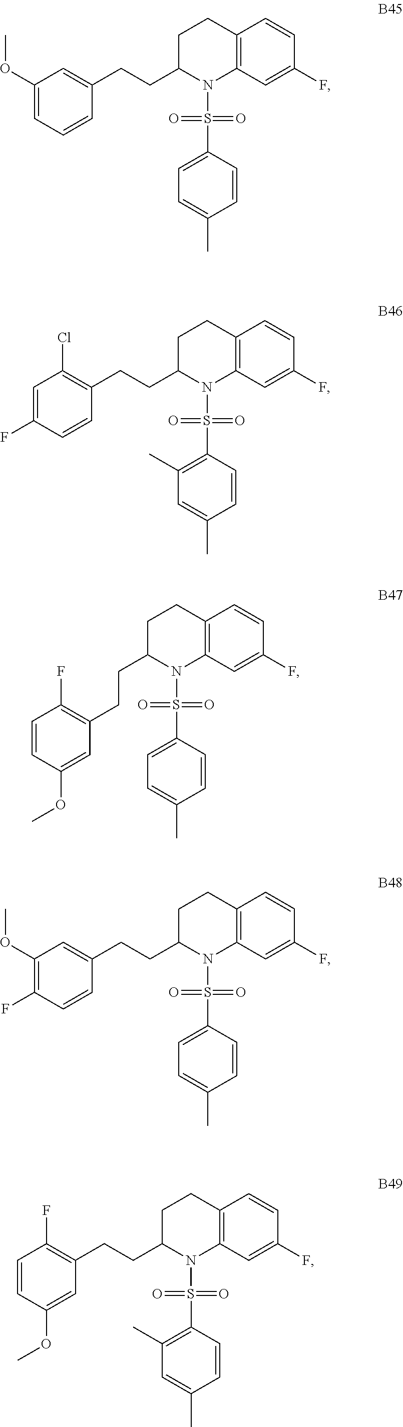 [SMILES: COc1cccc(CCC2CCc3ccc(F)cc3N2S(=O)(=O)c2ccc(C)cc2)c1 ; Cc1ccc(S(=O)(=O)N2c3cc(F)ccc3CCC2CCc2ccc(F)cc2Cl)c(C)c1 ; COc1ccc(F)c(CCC2CCc3ccc(F)cc3N2S(=O)(=O)c2ccc(C)cc2)c1 ; COc1cc(CCC2CCc3ccc(F)cc3N2S(=O)(=O)c2ccc(C)cc2)ccc1F ; COc1ccc(F)c(CCC2CCc3ccc(F)cc3N2S(=O)(=O)c2ccc(C)cc2C)c1]

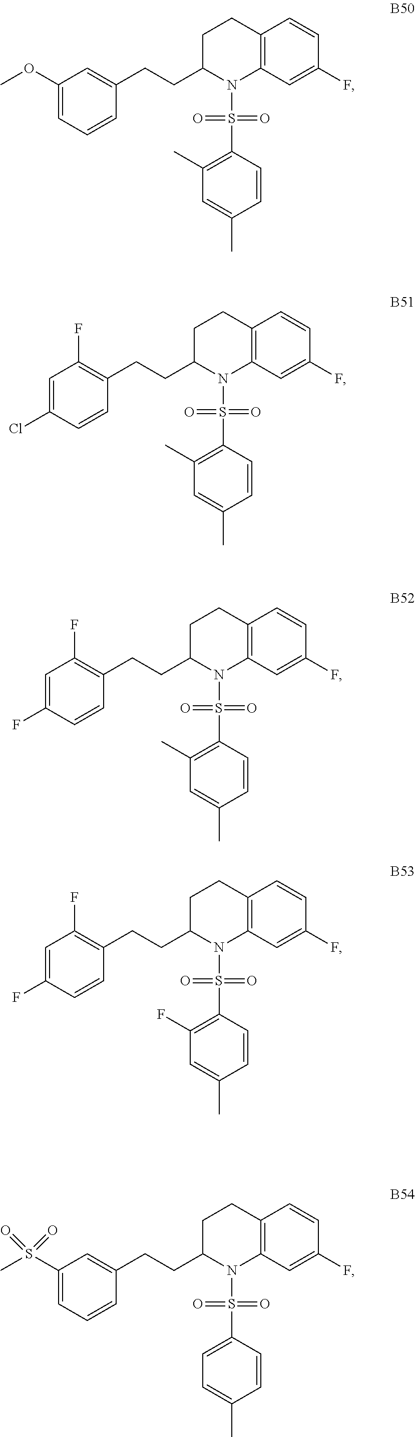

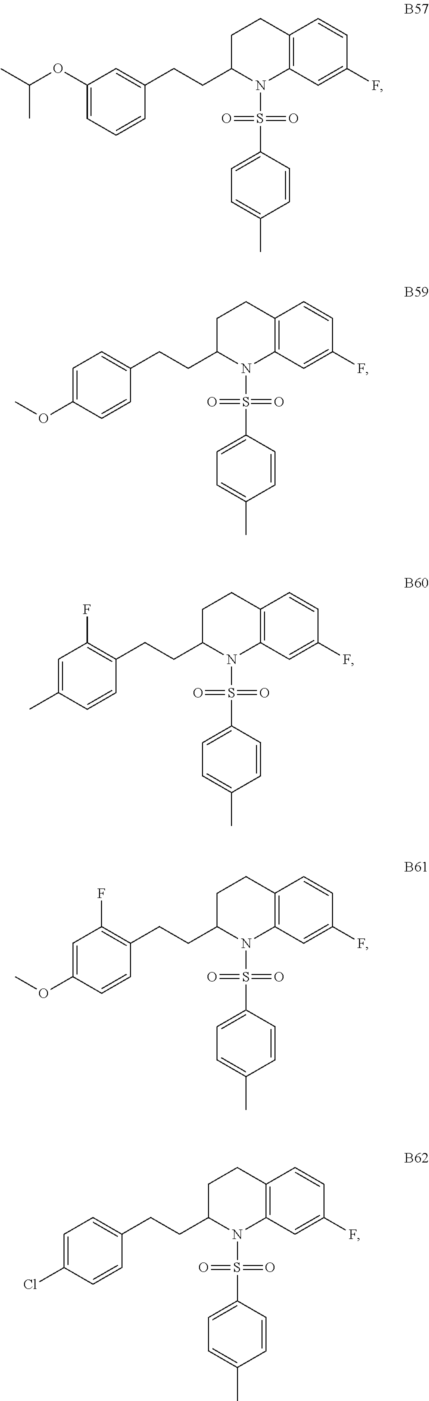 [SMILES: Cc1ccc(S(=O)(=O)N2c3cc(F)ccc3CCC2CCc2cccc(OC(C)C)c2)cc1 ; COc1ccc(CCC2CCc3ccc(F)cc3N2S(=O)(=O)c2ccc(C)cc2)cc1 ; Cc1ccc(S(=O)(=O)N2c3cc(F)ccc3CCC2CCc2ccc(C)cc2F)cc1 ; COc1ccc(CCC2CCc3ccc(F)cc3N2S(=O)(=O)c2ccc(C)cc2)c(F)c1 ; Cc1ccc(S(=O)(=O)N2c3cc(F)ccc3CCC2CCc2ccc(Cl)cc2)cc1]

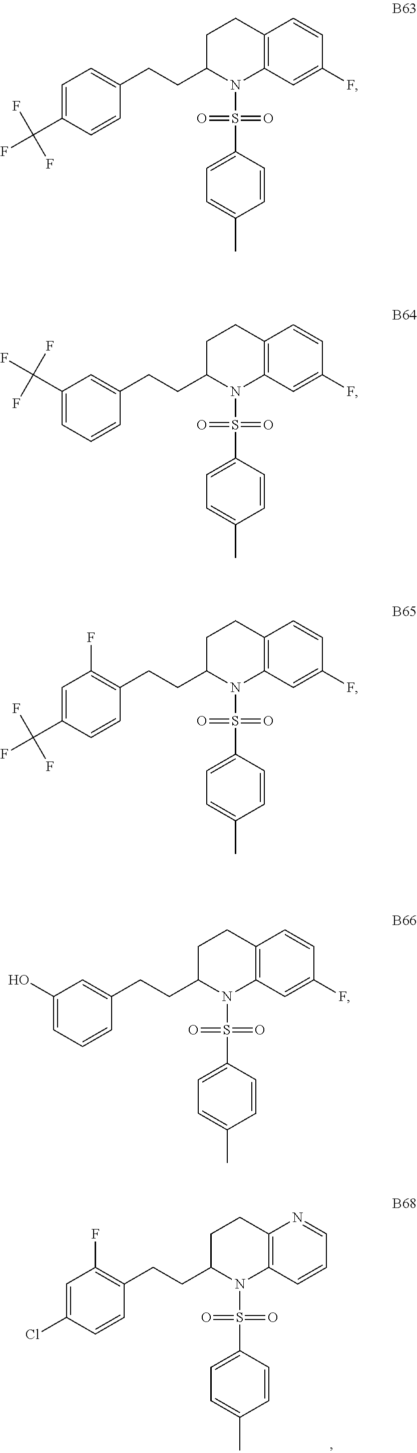 [SMILES: Cc1ccc(S(=O)(=O)N2c3cc(F)ccc3CCC2CCc2ccc(C(F)(F)F)cc2)cc1 ; Cc1ccc(S(=O)(=O)N2c3cc(F)ccc3CCC2CCc2cccc(C(F)(F)F)c2)cc1 ; Cc1ccc(S(=O)(=O)N2c3cc(F)ccc3CCC2CCc2ccc(C(F)(F)F)cc2F)cc1 ; Cc1ccc(S(=O)(=O)N2c3cc(F)ccc3CCC2CCc2cccc(O)c2)cc1 ; Cc1ccc(S(=O)(=O)N2c3cccnc3CCC2CCc2ccc(Cl)cc2F)cc1]

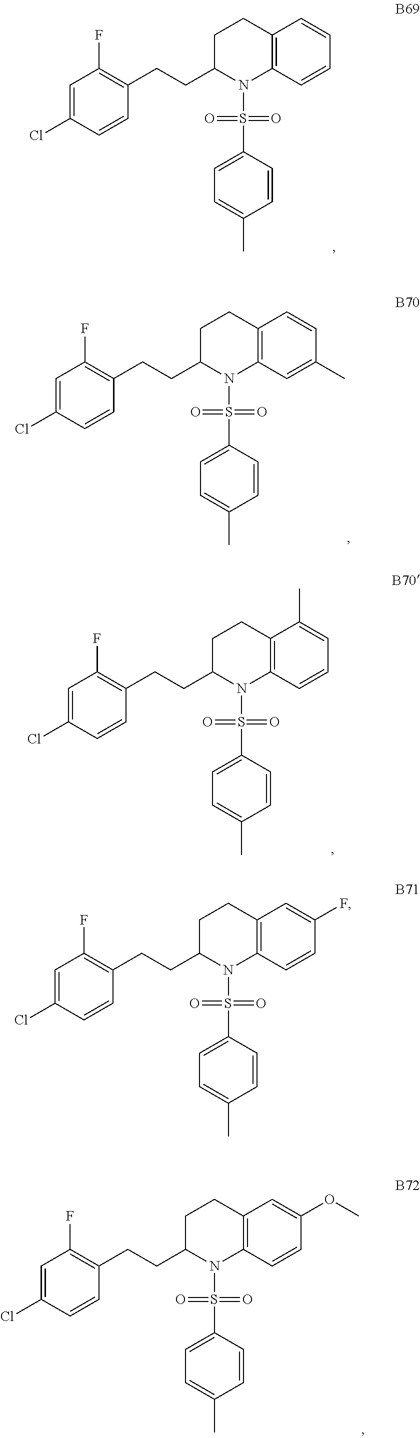 [SMILES: Cc1ccc(S(=O)(=O)N2c3ccccc3CCC2CCc2ccc(Cl)cc2F)cc1 ; Cc1ccc(S(=O)(=O)N2c3cc(C)ccc3CCC2CCc2ccc(Cl)cc2F)cc1 ; Cc1ccc(S(=O)(=O)N2c3cccc(C)c3CCC2CCc2ccc(Cl)cc2F)cc1 ; Cc1ccc(S(=O)(=O)N2c3ccc(F)cc3CCC2CCc2ccc(Cl)cc2F)cc1 ; COc1ccc2c(c1)CCC(CCc1ccc(Cl)cc1F)N2S(=O)(=O)c1ccc(C)cc1]

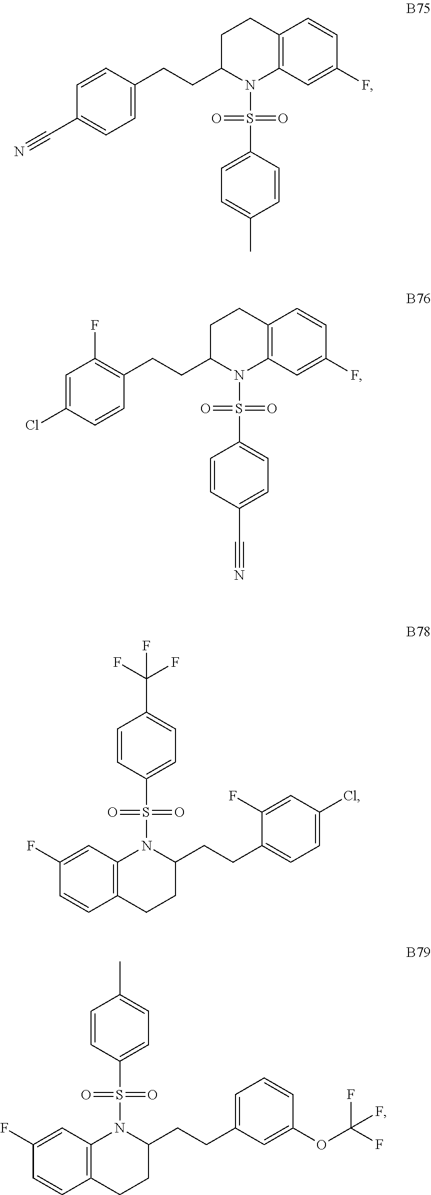 [SMILES: Cc1ccc(S(=O)(=O)N2c3cc(F)ccc3CCC2CCc2ccc(C#N)cc2)cc1 ; N#Cc1ccc(S(=O)(=O)N2c3cc(F)ccc3CCC2CCc2ccc(Cl)cc2F)cc1 ; O=S(=O)(c1ccc(C(F)(F)F)cc1)N1c2cc(F)ccc2CCC1CCc1ccc(Cl)cc1F ; Cc1ccc(S(=O)(=O)N2c3cc(F)ccc3CCC2CCc2cccc(OC(F)(F)F)c2)cc1]

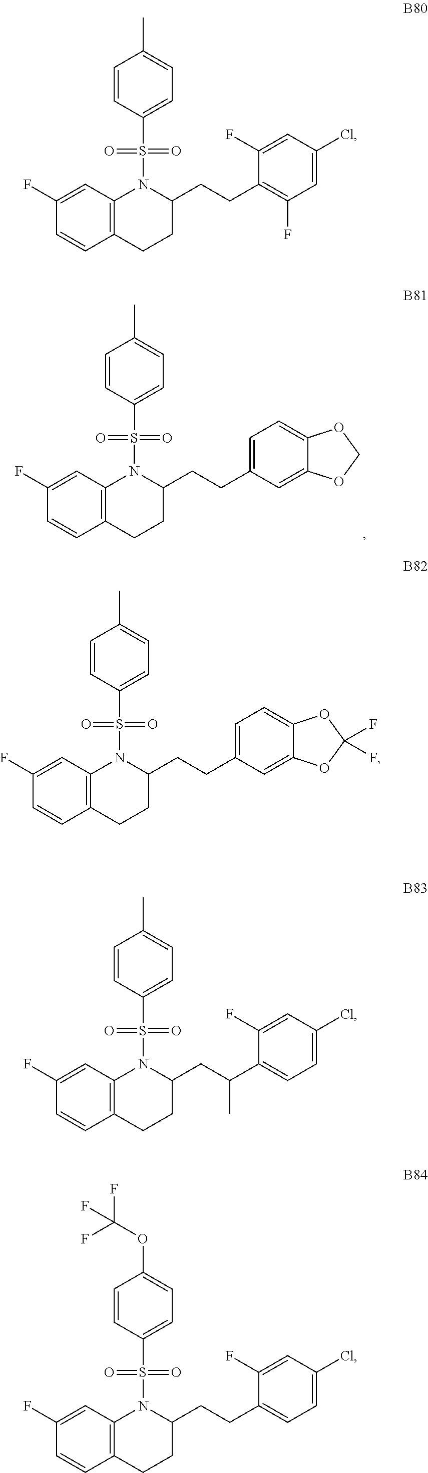 [SMILES: Cc1ccc(S(=O)(=O)N2c3cc(F)ccc3CCC2CCc2c(F)cc(Cl)cc2F)cc1 ; Cc1ccc(S(=O)(=O)N2c3cc(F)ccc3CCC2CCc2ccc3c(c2)OCO3)cc1 ; Cc1ccc(S(=O)(=O)N2c3cc(F)ccc3CCC2CCc2ccc3c(c2)OC(F)(F)O3)cc1 ; Cc1ccc(S(=O)(=O)N2c3cc(F)ccc3CCC2CC(C)c2ccc(Cl)cc2F)cc1 ; O=S(=O)(c1ccc(OC(F)(F)F)cc1)N1c2cc(F)ccc2CCC1CCc1ccc(Cl)cc1F]

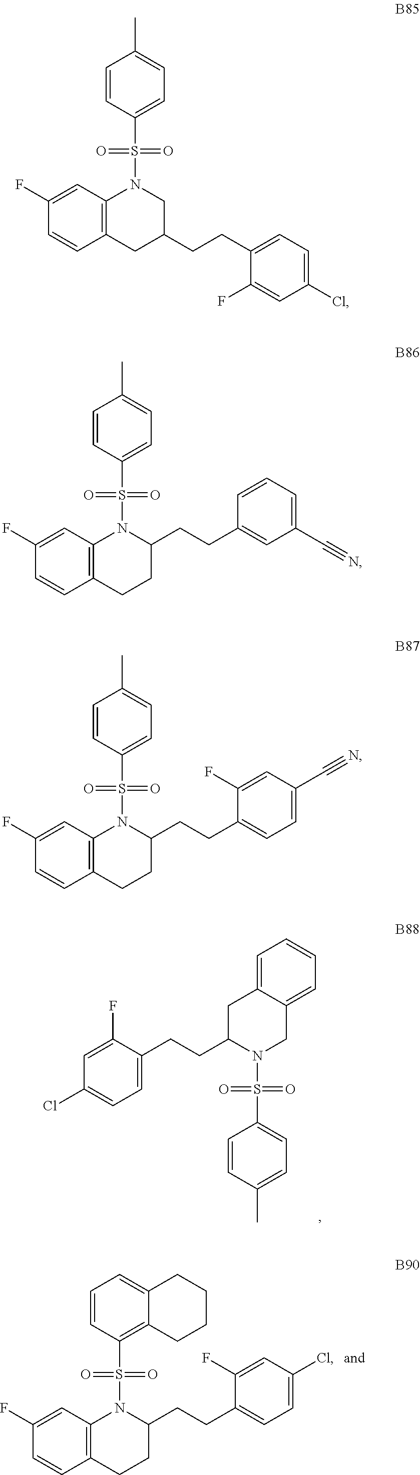 [SMILES: Cc1ccc(S(=O)(=O)N2CC(CCc3ccc(Cl)cc3F)Cc3ccc(F)cc32)cc1 ; Cc1ccc(S(=O)(=O)N2c3cc(F)ccc3CCC2CCc2cccc(C#N)c2)cc1 ; Cc1ccc(S(=O)(=O)N2c3cc(F)ccc3CCC2CCc2ccc(C#N)cc2F)cc1 ; Cc1ccc(S(=O)(=O)N2Cc3ccccc3CC2CCc2ccc(Cl)cc2F)cc1 ; O=S(=O)(c1cccc2c1CCCC2)N1c2cc(F)ccc2CCC1CCc1ccc(Cl)cc1F ; "and"]

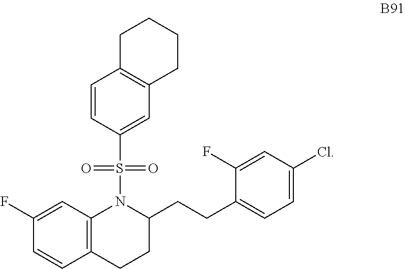 [SMILES: O=S(=O)(c1ccc2c(c1)CCCC2)N1c2cc(F)ccc2CCC1CCc1ccc(Cl)cc1F]

5. A compound of formula II: 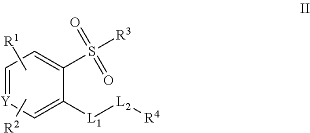
or a salt thereof; or a prodrug, or a salt of a prodrug thereof; or a hydrate, solvate, or polymorph thereof, wherein:
R¹ and R² each are independently H, alkyl, haloalkyl, alkoxy, heteroalkoxy, halogen, oxo, amino, or alkylamino; or R¹ and R² together with the atoms they are bonded form a 5-7 membered aryl, heteroaryl, cycloalkyl or partially unsaturated ring optionally substituted with one or more substituents independently selected from the group consisting of halo, cyano, (C₁-C₆)alkyl, (C₁-C₆)haloalkyl, (C₂-C₆)alkenyl, (C₂-C₆)alkynyl, (C₁-C₆)alkoxy, and (C₁-C₆)haloalkoxy;
R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, wherein any alkyl, haloalkyl, alkoxy, haloalkoxy, alkenyl, or alkynyl can be unsubstituted or substituted, each independently selected R′ is H or (C₁-C₆)alkyl or (C₃-C₇)cycloalkyl, and each independently selected R″ is (C₁-C₆)alkyl or (C₃-C₇)cycloalkyl;
Y is N or CR⁶; R⁶ is H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy;
L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR—; R is an (C₁-C₆)alkyl.

6. A compound according to claim 5, wherein said compound is of formula IIA: 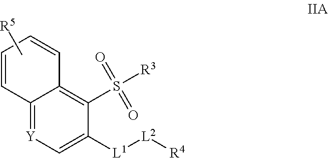
wherein R³ and R⁴ each are independently a 5-10 membered monocyclic or fused aryl or heteroaryl optionally substituted with one or more substituents independently selected from the group consisting of halo, cyano, hydroxyl, (C₁-C₆)alkyl, (C₁-C₆)haloalkyl, (C₂-C₆)alkenyl, (C₂-C₆)alkynyl, (C₁-C₆)alkoxy, (C₁-C₆)haloalkoxy, (C₁-C₆)alkoxycarbonyl, (C₃-C₇)cycloalkoxycarbonyl, R′NHC(═O), R′₂NC(═O), R″S, R″S(O), and R″S(O)₂, wherein any alkyl, haloalkyl, alkoxy, haloalkoxy, alkenyl, or alkynyl can be unsubstituted or substituted, and each independently selected R′ is H or (C₁-C₆)alkyl or (C₃-C₇)cycloalkyl, and each independently selected R″ is (C₁-C₆)alkyl or (C₃-C₇)cycloalkyl;
Y is N or CR⁶;
L¹ and L² are each independently a bond, (C₁-C₃)alkyl, —O—, —NH—, —S—, —S(O)—, —S(O)₂—, —NR—, or —C(O)—, provided L¹ and L² are not both —O—, —NH—, —S—, —S(O)—, —S(O)₂—, or —NR—; R is an (C₁-C₆)alkyl;
R⁵ and R⁶ are each independently H, halo, cyano, (C₁-C₆)alkyl, (C₁-C₆)haloalkyl, (C₂-C₆)alkenyl, (C₂-C₆)alkynyl, (C₁-C₆)alkoxy, or (C₁-C₆)haloalkoxy.

7. A compound according to claim 5, wherein said compound is selected from the group consisting of:

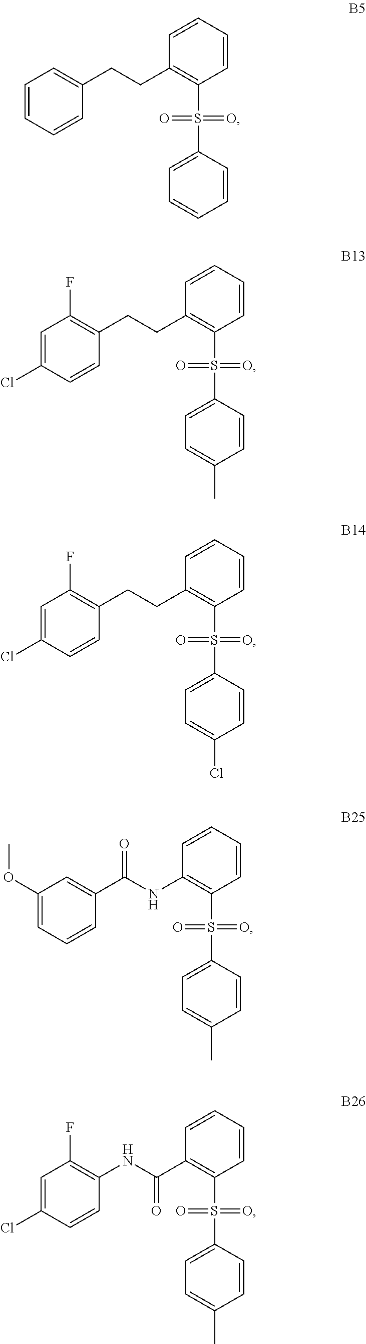

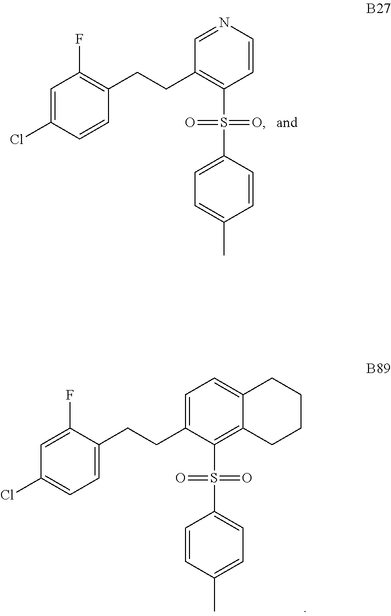 [SMILES: Cc1ccc(S(=O)(=O)c2ccncc2CCc2ccc(Cl)cc2F)cc1 ; "and" ; Cc1ccc(S(=O)(=O)c2c(CCc3ccc(Cl)cc3F)ccc3c2CCCC3)cc1]

8. A compound according to claim 6, wherein said compound is selected from the group consisting of:

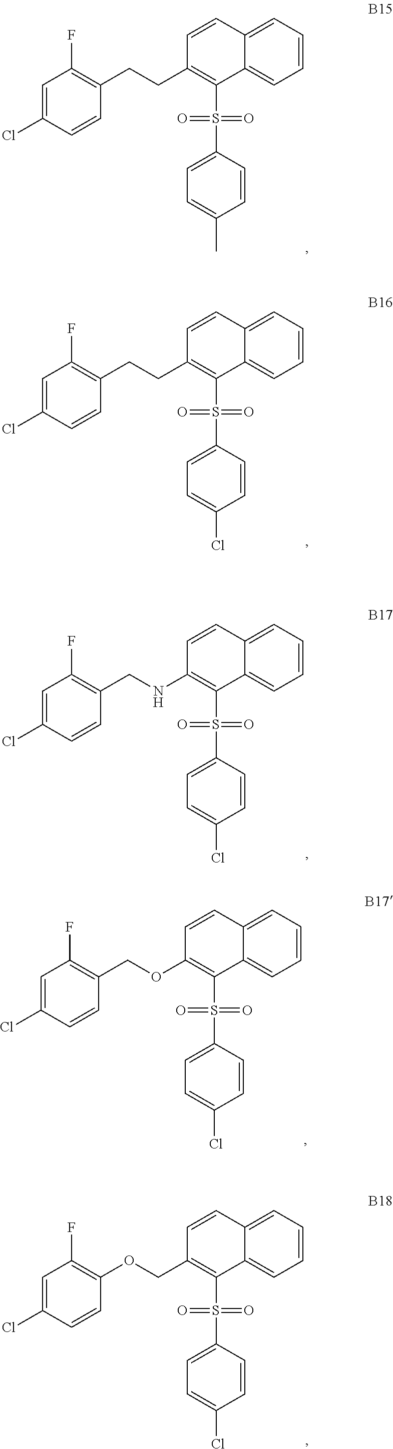 [SMILES: Cc1ccc(S(=O)(=O)c2c(CCc3ccc(Cl)cc3F)ccc3ccccc23)cc1 ; O=S(=O)(c1ccc(Cl)cc1)c1c(CCc2ccc(Cl)cc2F)ccc2ccccc12 ; O=S(=O)(c1ccc(Cl)cc1)c1c(NCc2ccc(Cl)cc2F)ccc2ccccc12 ; O=S(=O)(c1ccc(Cl)cc1)c1c(OCc2ccc(Cl)cc2F)ccc2ccccc12 ; O=S(=O)(c1ccc(Cl)cc1)c1c(COc2ccc(Cl)cc2F)ccc2ccccc12]

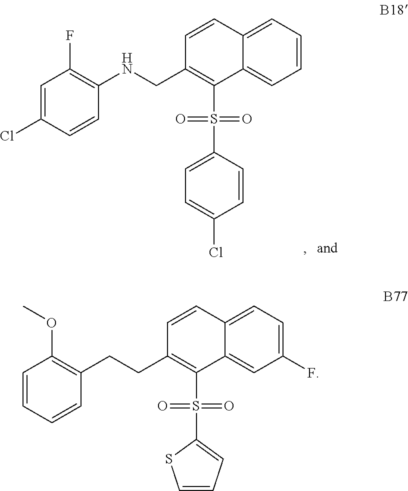 [SMILES: O=S(=O)(c1ccc(Cl)cc1)c1c(CNc2ccc(Cl)cc2F)ccc2ccccc12 ; COc1ccccc1CCc1ccc2ccc(F)cc2c1S(=O)(=O)c1cccs1]

9. A method for modulating TRPMLs in a mammal, comprising administering to the mammal an effective amount of a compound of claim 1.

10. A method for treating a condition in a mammal, wherein modulation of TRPMLs is medically indicated, comprising administering to the mammal an effective amount of a compound of claim 1.

11. The method of claim 10 wherein the condition is an acid-related disorder.

12. The method of claim 11 wherein the condition is a gastric disorder.

13. The method of treating an acid-related disorder by combining a compound of claim 1 with another agent.

14. The method of claim 13 where the other agent is a proton pump inhibitor.

15. A method for modulating lysosome function in a mammal, comprising administering to the mammal an effective amount of a compound of claim 1.

16. A method for treating a condition in a mammal, wherein abnormal functioning of lysosomes is medically indicated, comprising administering to the mammal an effective amount of a compound of claim 1.

17. The method of claim 16 wherein the condition is cancer.

18. A method for treating an acid-related disorder using a TRPML inhibitor.

19. A method for modulating tubulovesicle and lysosome functions in a mammal, comprising administering to the mammal an effective amount of a TRPML inhibitor.

20. A method for treating a condition in a mammal, wherein abnormal functioning of lysosomes is medically indicated, comprising administering to the mammal an effective amount of a TRPML inhibitor.
